SEQUENCE LISTING (27 sequences)

SEQ ID NO: 1 - DNA, 7355 bp, Artificial Sequence
  synthetic
ctggcgaaagggggatgtgctgcaaggcgattaagttgggtaacgccagggttttcccagtcacgacgttgtaaaacgacggccagtgaattcgttaaccccatgcactcaactatccccgtaaacatagtagtaggctataggctttcttctcgtcatcacagagaagccatatagagacatatacatattaatacatggacttctaggcttactttctcacatacttttcacgtactttctcacatatttactaatacacaatttttacattatgtgcatttgtaaattgcaatgtaggattttcctcattgacaaggacaaaccctcatttgggaggaaaagagaaaataacttctccgtttttttttgtttttttttctcagatgagccgccactgttttttttttttttgcatccccctttttccaatttgtcatgactataaaagcgcgaattccccccccccccttgtctcccgtttctttcctgccatgcgagattgctcattcaatttgaccttctcttgatgtggcaatttatctccaaaactgacagcttaagcttctttccaatgttggtttctggtaaacgaatgccatagagaaaggctggagatacagagacaggtctgtggttgtttcccacacgggtttgtttttgattctcctcgacatggtttgtctgctgaatatatataaatattcatacacctctatctaactatctatctataaaaaatcgcaatgtagtagctatattaacattaactatggcaataataacaatagcaatactgaaaacaatgatgttattattattatcgtgggcaataccaaaaaccgaggtcattcaaaagggcgcacattggtggaaaaacaatacttgcgcggaaaaaaaaaaattaatatctgccaaatcctaaactgcaaatcatggattgcaattctccgttatctatataaggctaggaggttgccaatcggtatataatagttgggggggaggaggaggaggattctctttagattatcacttgatacaaatcaatcggttatcttttattgaaaagtcaagacacaaaccaaccaactaaacgagcggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaagctagctaaaatgagagttgattccaagatcattgtcaagaaagagactcaagacgaaaacttgtatcaatccttggccgaaagatccaagcacgaggagtttcttagaaaggcagtcgatctgttggttgagagagttgtttttggtagatctactagatcttctaaggtcgttgaatgggctgcaccagacgaaatcaagaaagcaattgatttgaagcctagattaggtccagcttcccatgatgaactcttggcgttcatggcaaatgtcgctagatattctgttaacactggtcatccatacttcgttaaccaacttttctcttcagttgatccatatggtttggtcggtcaatggttaaccgacgccttaaacccatccgtttacaccttcgaagtcgcacctgtcttcactttgatggaagaagaagttttgagggaaatgagaaagatcgtcggttggccagaaggtgaaggtgatggcattttctgtccaggtggttccatcgcaaatggttacgcaatttcttgtgcaagacaccacttttacccagaagttaagtacaaaggtgttcacgctgttcctaagttggttttgttcacgtccgagcttgctcactattccaccaaaaagatggcagcattcatgggtattggctccgacaattgtgtcaatatcaaaaccgatgatgttggtaagatgaacatcgttgatttggaaatgaagatcaagatcgcaattgataacaagtgtactccattcatggttaccgcaaccagtggtaccactgtctttggtgcattcgaccctttggttgcaattagtgacttatgtaagaagtataacttatggttgcacgttgacgctgcttggggtggtggtgccttgatgtctaagaagcatagacacttgttgaatggcattgaattggcagattctgttacttggaatccacataagttgcttgccgctccacaacaatgttctaccttcttaaccagacataagaaagtcttatctgaaggtcacagttccaatgccaagtatctgtttcaaaaggataagttctacgatacatcttacgacactggcgacaaacatattcaatgtggtagaagagcagacgtcttgaagttttggttcatgtggaaggcaaaaggtacagaaggtttcgaaaagcatgttgacaagctattcgacaacgcgaagtactttttggatcacatcaaacaaagagaaggttttcagctagttatcgctgaaccacaatgtaccaacattatgttctggtacattccaaagtgtctccgtggttgtgaaaacgatgcagattactatgagagactccacaaggtcgcacctaagatcaaagaacgtatgatcaaagaaggttctatgatggtcacctaccaaccacaaggtgacttagttaacttctttagaatcgttttccaaaactccgcattagatcacaaggatatggtttactttgcaaatgagttcgaaagattaggttccgacatgattgtttaaggcgcgccttctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaaggaggggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaaactagttaaaatgagagttgactcaaagatcattgtcaagaaggaaacacaagatgaaaacctctaccaatctttagcagaaagatccaaacatgaagagttcttaagaaaggccgttgacttacttgtcgaaagagttgttttcggcagatcaaccagatcttccaaggttgttgagtgggctgcaccagacgagattaagaaagcaattgacttaaagccacgtttgggtcctgcttctcacgatgagttgttggcctttatggcaaacgttgctaggtattctgtcaatactggtcatccatacttcgttaaccagttattctcatccgttgatccatatggtttagttggtcaatggttaaccgacgcattgaacccatccgtttacacctttgaagtcgctcctgtctttaccttgatggaggaagaggttttgcgtgaaatgcgtaagattgtcggttggcctgagggcgaaggtgatggtatcttctgcccaggtggttctatcgccaacggttacgctatttcctgtgctagacatcacttttacccagaggtcaagtataagggtgttcatgctgttcctaagttagttttgtttacctccgaactcgcgcactacagtaccaaaaagatggctgctttcatgggtattggttccgataactgtgttaacattaagaccgatgatgttggtaagatgaacatcgttgatttagaaatgaagatcaagattgctattgacaacaagtgtaccccattcatggttacagctacctctggtactaccgttttcggtgctttcgacccattagtcgcaatctccgatctttgcaagaagtacaatctgtggttgcatgtcgatgctgcttggggtggtggtgctctcatgtctaagaagcacagacaccttttgaacggtatcgaattagccgactctgttacttggaaccctcataagcttttagccgctccacaacaatgttccacctttttgaccagacacaagaaagttctatccgaaggtcattcatccaatgcaaagtacttattccaaaaagacaagttttacgacacatcctacgacactggtgataaacatattcaatgtggtagaagagcagatgttcttaagttctggttcatgtggaaggctaagggtactgaaggtttcgagaaacacgttgacaagctttttgataacgcaaagtacttcttagaccacattaagcagagagaaggtttccaattggttattgcagaaccacaatgcaccaatatcatgttctggtacatcccaaagtgcctgagaggttgtgaaaatgatgcagactactatgagagattacataaagttgcgcctaagatcaaggaaagaatgatcaaggaaggttcaatgatggtcacctaccaaccacaaggtgatcttgttaacttctttagaattgtttttcagaactctgcattggaccataaggacatggtttacttcgcaaatgaattcgaaagattgggctccgatatgatcgtttaattaattaatttattttactagtttatttttgctcctgagaataggattacaaacacttaaagtctttaattacaactatatataatattctgttggttttcttgaattggttcgctgcgattcatgcctcccattcaccaaaggtggagtgggaaataacggttttactgcggtaattagcagaggcaagaacaggatacactttttgatgataaatctgtattatagtcgagcctatttaggaaatcaaattttcttgtgtttacttttcaaataaataatgttcgaaaatttttactttactccttcatttaactataccagacgttatatcatcaacaccttctgaccatatacagctcaagatgtttaagagtctgttaaattttttcaatccatttcatggagtaccaggaggtgctacaaaaggaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactccgc

SEQ ID NO: 2 - DNA, 7997 bp, Artificial Sequence
  synthetic
ctggcgaaagggggatgtgctgcaaggcgattaagttgggtaacgccagggttttcccagtcacgacgttgtaaaacgacggccagtgaattctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacagctagctaaaatgcgtgttgactccaagattatcgttaagaaagaaacccaagatgagaacttgtatcaatctttggcagaaagatccaagcatgaagagtttttgcgtaaagcagttgatctgttagttgaaagagttgtcttcggtagatctacaagatcttcaaaagttgttgaatgggctgcaccagatgagattaagaaagcaattgacttaaagccaagattaggccctgcatctcatgacgaattactagcctttatggcaaatgtcgctaggtatagtgtcaatactggtcacccatacttcgttaatcaattgttttcttccgtcgatccatatggtttggtcggtcaatggttgaccgacgcgctcaatccaagtgtttacacctttgaagttgcaccagtctttacgctcatggaagaggaagtcttgagagaaatgaggaagattgttggttggcctgaaggtgagggcgacggtatcttctgtccaggcggttctattgcaaacggttacgcaatttcttgtgctagacaccacttctacccagaggttaagtacaagggtgttcacgcagttccaaagttggtcttgtttacctcagaattggcacactattctacgaagaagatggctgcctttatgggtattggtagtgataactgtgttaacatcaaaaccgatgacgttggtaagatgaacattgttgatttagaaatgaagattaagatcgcaattgataacaagtgtactccattcatggttactgctacttccggtacaacggttttcggtgcttttgatccattagttgctatttccgatttgtgtaagaaatacaatctatggcttcatgttgatgctgcgtggggtggtggtgctttgatgtccaagaaacacagacacttattgaacggtattgaattagccgactctgtcacttggaatcctcataagttgttagcagctccacagcaatgttccaccttcttaactagacacaagaaagtcttgtccgaaggccacagttccaacgcaaagtacttgttccaaaaggacaaattctacgatacctcttacgacactggcgataagcatatccaatgtggtagaagagcagatgttctgaagttttggttcatgtggaaagctaagggtactgaaggtttcgagaaacacgtcgataaactattcgataacgccaagtatttcttggatcatatcaagcaaagagagggtttccaattagtcattgcagagcctcaatgtaccaatatcatgttctggtacattccaaagtgccttagaggttgtgaaaacgatgcagattactatgaaagactacataaggttgcgccaaagatcaaggaacgtatgattaaggaaggttctatgatggttacctaccaaccacaaggtgatttggttaacttcttcagaatcgtcttccagaactctgctcttgatcacaaggatatggtttacttcgccaacgaatttgaacgtttgggttccgatatgattgtttaaggcgcgccatctaatagtttaatcacagcttatagtctattatagttttcttttttaaacattgttgtattttgtccccccccctctaattgatgatgattatcctataagaatccaataaaacgatggaaactaatactttctcctttgtcatgtggtctttagtatttcttgaacattggctctgatttctcgactttatagtcctattaaaatctctgttagttctcgatcgttgtatctcgtttcttgtctctttggtggatgattttgcgtgcgaacatgtttttttccctttctctcaccatcatcgtgtagttcttgtcaccatccccccaccccttccttctctcattgattctataagagcttatccacagaggtgcagtaacgaggtagtttaaccttcgagtggatcaaaatgtcacacaggaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacatctagataaaatgagggttgactccaaaatcatcgtcaaaaaggaaacccaagatgaaaacttgtaccaatctttggctgaaagatccaagcatgaagagtttttgagaaaagccgttgatttgttggttgaaagagttgtttttggtagatccactagatcctccaaggtcgttgaatgggcagctccagatgaaatcaaaaaggccatcgatcttaagcctaggttaggcccagcttcccatgatgaattgttagcctttatggcaaatgttgcaagatattccgtcaatactggtcacccatacttcgttaatcaattgttttcatctgttgatccatacggtctagttggtcaatggttgaccgatgccttaaacccttctgtctatacctttgaagttgctccagtctttacattgatggaggaagaggttttgagagaaatgaggaaaatcgttggttggccagaaggtgagggtgatggcatcttttgtccaggtggttctatcgccaacggttacgctatttcttgtgctcgtcatcacttctacccagaagttaagtacaaaggtgttcatgctgtccctaagttagtcttgtttacctctgaattagctcactattctacaaagaaaatggctgcattcatgggtattggttccgacaattgtgtcaatatcaaaacagacgacgttggtaagatgaacattgttgacctagaaatgaagattaagatcgcaattgacaacaagtgtacccctttcatggttacagctacatcaggtactactgttttcggtgcttttgacccacttgtcgcaatctccgatttgtgtaagaagtacaacttatggctacatgtcgatgcggcatggggtggtggtgcgttaatgtcaaagaagcatagacatcttctaaacggtatcgaattagcagactccgttacctggaatccacacaaattgttagctgctcctcaacaatgttccacattccttaccagacacaagaaggtcctgtctgaaggtcattcttccaacgccaaatacttgtttcagaaggacaagttttacgacacttcttatgacactggtgacaaacatattcagtgtggtagaagagcagatgttttgaagttctggtttatgtggaaggcaaagggtacggagggtttcgagaagcacgttgataagttgtttgacaatgctaagtacttcctagatcacattaagcaaagggaaggtttccaattggtcattgcagaacctcagtgtaccaacatcatgttctggtatatcccaaagtgtctaagaggttgtgaaaatgatgcagactactatgaaagattgcataaagtcgctccaaagatcaaagaaagaatgatcaaagaaggttctatgatggtcacctaccaaccacaaggtgatttggtcaatttctttagaatcgttttccagaattctgctttagatcataaggatatggtctactttgcaaacgaattcgaaagattaggttccgacatgatcgtttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagcggccgccaacttggttcaaaccaagaaagccgtcgtgtactttgcgtattttcaacttcttcccttcaactccaaccttataacccaatccgacagttagcatacagtagaaaatccataagttaattgcaaaaaccacataattcccaatgactggcacaatttgcatgaacggcgcaaaaccaacacctatccttcttgacttgtggacaacttctccggcatcggctccagtttctggtttaggcgacactttcaaggtacccttgttgactaggttctcagcaacatactcctcctcgataactttgaccgtctttgtgccaaacacattgttcaatagtttggacgctcgcataccgaagacagactcgtccatccaatagcatgtactccgaatgctctcaatcaacggtatcaaatcgcttggtgtctttggaggcggtgttctaggtttcatcttcgccttcccgtttcctgtttcttggacttcaaaatacgggtcatatgcatcttcattggtgaacctgaacagtttgggccgagtagtgccaaacaccttcaacagtataatcttcactccttggccgattagaaacatgcttatatgtttgccggtattcttaaagtgtatactggagcccttttaatcacattttttttacttgtcagtctccatacggagtttaatgtccttatatcgatcttcatcagctccaacgggacaggaatcaataaccttccttgcctgtcccaaaaagaatgaattttttttcaaaagctttacgatgcataccacaaaaggaagattattcccacatgttccagaagtgtgcggagatacaaagggttcatgaaaacgtgaatcttctaaaaacttagcacaacaataaaaatctacaatgttacagtaagtattattttctttttgtcgacacactccaacggttagatttccaagtattcaatccaatgtattacttgtcagacagccatccactcccatcttagaacatcacttccgaaccgc

SEQ ID NO: 3 - DNA, 7859 bp, Artificial Sequence
  synthetic
ctggcgaaagggggatgtgctgcaaggcgattaagttgggtaacgccagggttttcccagtcacgacgttgtaaaacgacggccagtgaattcgttaaccccatgcactcaactatccccgtaaacatagtagtaggctataggctttcttctcgtcatcacagagaagccatatagagacatatacatattaatacatggacttctaggcttactttctcacatacttttcacgtactttctcacatatttactaatacacaatttttacattatgtgcatttgtaaattgcaatgtaggattttcctcattgacaaggacaaaccctcatttgggaggaaaagagaaaataacttctccgtttttttttgtttttttttctcagatgagccgccactgttttttttttttttgcatccccctttttccaatttgtcatgactataaaagcgcgaattccccccccccccttgtctcccgtttctttcctgccatgcgagattgctcattcaatttgaccttctcttgatgtggcaatttatctccaaaactgacagcttaagcttctttccaatgttggtttctggtaaacgaatgccatagagaaaggctggagatacagagacaggtctgtggttgtttcccacacgggtttgtttttgattctcctcgacatggtttgtctgctgaatatatataaatattcatacacctctatctaactatctatctataaaaaatcgcaatgtagtagctatattaacattaactatggcaataataacaatagcaatactgaaaacaatgatgttattattattatcgtgggcaataccaaaaaccgaggtcattcaaaagggcgcacattggtggaaaaacaatacttgcgcggaaaaaaaaaaattaatatctgccaaatcctaaactgcaaatcatggattgcaattctccgttatctatataaggctaggaggttgccaatcggtatataatagttgggggggaggaggaggaggattctctttagattatcacttgatacaaatcaatcggttatcttttattgaaaagtcaagacacaaaccaaccaactaaacgagcggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaagctagctaaaatgagagttgattccaagatcattgtcaagaaagagactcaagacgaaaacttgtatcaatccttggccgaaagatccaagcacgaggagtttcttagaaaggcagtcgatctgttggttgagagagttgtttttggtagatctactagatcttctaaggtcgttgaatgggctgcaccagacgaaatcaagaaagcaattgatttgaagcctagattaggtccagcttcccatgatgaactcttggcgttcatggcaaatgtcgctagatattctgttaacactggtcatccatacttcgttaaccaacttttctcttcagttgatccatatggtttggtcggtcaatggttaaccgacgccttaaacccatccgtttacaccttcgaagtcgcacctgtcttcactttgatggaagaagaagttttgagggaaatgagaaagatcgtcggttggccagaaggtgaaggtgatggcattttctgtccaggtggttccatcgcaaatggttacgcaatttcttgtgcaagacaccacttttacccagaagttaagtacaaaggtgttcacgctgttcctaagttggttttgttcacgtccgagcttgctcactattccaccaaaaagatggcagcattcatgggtattggctccgacaattgtgtcaatatcaaaaccgatgatgttggtaagatgaacatcgttgatttggaaatgaagatcaagatcgcaattgataacaagtgtactccattcatggttaccgcaaccagtggtaccactgtctttggtgcattcgaccctttggttgcaattagtgacttatgtaagaagtataacttatggttgcacgttgacgctgcttggggtggtggtgccttgatgtctaagaagcatagacacttgttgaatggcattgaattggcagattctgttacttggaatccacataagttgcttgccgctccacaacaatgttctaccttcttaaccagacataagaaagtcttatctgaaggtcacagttccaatgccaagtatctgtttcaaaaggataagttctacgatacatcttacgacactggcgacaaacatattcaatgtggtagaagagcagacgtcttgaagttttggttcatgtggaaggcaaaaggtacagaaggtttcgaaaagcatgttgacaagctattcgacaacgcgaagtactttttggatcacatcaaacaaagagaaggttttcagctagttatcgctgaaccacaatgtaccaacattatgttctggtacattccaaagtgtctccgtggttgtgaaaacgatgcagattactatgagagactccacaaggtcgcacctaagatcaaagaacgtatgatcaaagaaggttctatgatggtcacctaccaaccacaaggtgacttagttaacttctttagaatcgttttccaaaactccgcattagatcacaaggatatggtttactttgcaaatgagttcgaaagattaggttccgacatgattgtttaaggcgcgccttctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaaggaggggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaaactagttaaaatgagagttgactcaaagatcattgtcaagaaggaaacacaagatgaaaacctctaccaatctttagcagaaagatccaaacatgaagagttcttaagaaaggccgttgacttacttgtcgaaagagttgttttcggcagatcaaccagatcttccaaggttgttgagtgggctgcaccagacgagattaagaaagcaattgacttaaagccacgtttgggtcctgcttctcacgatgagttgttggcctttatggcaaacgttgctaggtattctgtcaatactggtcatccatacttcgttaaccagttattctcatccgttgatccatatggtttagttggtcaatggttaaccgacgcattgaacccatccgtttacacctttgaagtcgctcctgtctttaccttgatggaggaagaggttttgcgtgaaatgcgtaagattgtcggttggcctgagggcgaaggtgatggtatcttctgcccaggtggttctatcgccaacggttacgctatttcctgtgctagacatcacttttacccagaggtcaagtataagggtgttcatgctgttcctaagttagttttgtttacctccgaactcgcgcactacagtaccaaaaagatggctgctttcatgggtattggttccgataactgtgttaacattaagaccgatgatgttggtaagatgaacatcgttgatttagaaatgaagatcaagattgctattgacaacaagtgtaccccattcatggttacagctacctctggtactaccgttttcggtgctttcgacccattagtcgcaatctccgatctttgcaagaagtacaatctgtggttgcatgtcgatgctgcttggggtggtggtgctctcatgtctaagaagcacagacaccttttgaacggtatcgaattagccgactctgttacttggaaccctcataagcttttagccgctccacaacaatgttccacctttttgaccagacacaagaaagttctatccgaaggtcattcatccaatgcaaagtacttattccaaaaagacaagttttacgacacatcctacgacactggtgataaacatattcaatgtggtagaagagcagatgttcttaagttctggttcatgtggaaggctaagggtactgaaggtttcgagaaacacgttgacaagctttttgataacgcaaagtacttcttagaccacattaagcagagagaaggtttccaattggttattgcagaaccacaatgcaccaatatcatgttctggtacatcccaaagtgcctgagaggttgtgaaaatgatgcagactactatgagagattacataaagttgcgcctaagatcaaggaaagaatgatcaaggaaggttcaatgatggtcacctaccaaccacaaggtgatcttgttaacttctttagaattgtttttcagaactctgcattggaccataaggacatggtttacttcgcaaatgaattcgaaagattgggctccgatatgatcgtttaattaattaatttattttactagtttatttttgctcctgagaataggattacaaacacttaaagtctttaattacaactatatataatattctgttggttttcttgaattggttcgctgcgattcatgcctcccattcaccaaaggtggagtgggaaataacggttttactgcggtaattagcagaggcaagaacaggatacactttttgatgataaatctgtattatagtcgagcctatttaggaaatcaaattttcttgtgtttacttttcaaataaataatgttcgaaaatttttactttactccttcatttaactataccagacgttatatcatcaacaccttctgaccatatacagctcaagatgtttaagagtctgttaaattttttcaatccatttcatggcctgcagggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgaaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaatctagatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgc

SEQ ID NO: 4 - DNA, 7358 bp, Artificial Sequence
  synthetic
ggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagaaattcggatccggtagatacattgatgctatcaatccagagaactggaaagattgtgtagccttgaaaaacggtgaaacttacgggtccaagattgtctacagattttcctgatttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccataacttcgtataatgtatgctatacgaagttattaattaacaaacagcaatttgaggaaggaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacagctagctaaaatgcgtgttgactccaagattatcgttaagaaagaaacccaagatgagaacttgtatcaatctttggcagaaagatccaagcatgaagagtttttgcgtaaagcagttgatctgttagttgaaagagttgtcttcggtagatctacaagatcttcaaaagttgttgaatgggctgcaccagatgagattaagaaagcaattgacttaaagccaagattaggccctgcatctcatgacgaattactagcctttatggcaaatgtcgctaggtatagtgtcaatactggtcacccatacttcgttaatcaattgttttcttccgtcgatccatatggtttggtcggtcaatggttgaccgacgcgctcaatccaagtgtttacacctttgaagttgcaccagtctttacgctcatggaagaggaagtcttgagagaaatgaggaagattgttggttggcctgaaggtgagggcgacggtatcttctgtccaggcggttctattgcaaacggttacgcaatttcttgtgctagacaccacttctacccagaggttaagtacaagggtgttcacgcagttccaaagttggtcttgtttacctcagaattggcacactattctacgaagaagatggctgcctttatgggtattggtagtgataactgtgttaacatcaaaaccgatgacgttggtaagatgaacattgttgatttagaaatgaagattaagatcgcaattgataacaagtgtactccattcatggttactgctacttccggtacaacggttttcggtgcttttgatccattagttgctatttccgatttgtgtaagaaatacaatctatggcttcatgttgatgctgcgtggggtggtggtgctttgatgtccaagaaacacagacacttattgaacggtattgaattagccgactctgtcacttggaatcctcataagttgttagcagctccacagcaatgttccaccttcttaactagacacaagaaagtcttgtccgaaggccacagttccaacgcaaagtacttgttccaaaaggacaaattctacgatacctcttacgacactggcgataagcatatccaatgtggtagaagagcagatgttctgaagttttggttcatgtggaaagctaagggtactgaaggtttcgagaaacacgtcgataaactattcgataacgccaagtatttcttggatcatatcaagcaaagagagggtttccaattagtcattgcagagcctcaatgtaccaatatcatgttctggtacattccaaagtgccttagaggttgtgaaaacgatgcagattactatgaaagactacataaggttgcgccaaagatcaaggaacgtatgattaaggaaggttctatgatggttacctaccaaccacaaggtgatttggttaacttcttcagaatcgtcttccagaactctgctcttgatcacaaggatatggtttacttcgccaacgaatttgaacgtttgggttccgatatgattgtttaaggcgcgccatctaatagtttaatcacagcttatagtctattatagttttcttttttaaacattgttgtattttgtccccccccctctaattgatgatgattatcctataagaatccaataaaacgatggaaactaatactttctcctttgtcatgtggtctttagtatttcttgaacattggctctgatttctcgactttatagtcctattaaaatctctgttagttctcgatcgttgtatctcgtttcttgtctctttggtggatgattttgcgtgcgaacatgtttttttccctttctctcaccatcatcgtgtagttcttgtcaccatccccccaccccttccttctctcattgattctataagagcttatccacagaggtgcagtaacgaggtagtttaaccttcgagtggatcaaaatgtcacacaggaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacatctagataaaatgagggttgactccaaaatcatcgtcaaaaaggaaacccaagatgaaaacttgtaccaatctttggctgaaagatccaagcatgaagagtttttgagaaaagccgttgatttgttggttgaaagagttgtttttggtagatccactagatcctccaaggtcgttgaatgggcagctccagatgaaatcaaaaaggccatcgatcttaagcctaggttaggcccagcttcccatgatgaattgttagcctttatggcaaatgttgcaagatattccgtcaatactggtcacccatacttcgttaatcaattgttttcatctgttgatccatacggtctagttggtcaatggttgaccgatgccttaaacccttctgtctatacctttgaagttgctccagtctttacattgatggaggaagaggttttgagagaaatgaggaaaatcgttggttggccagaaggtgagggtgatggcatcttttgtccaggtggttctatcgccaacggttacgctatttcttgtgctcgtcatcacttctacccagaagttaagtacaaaggtgttcatgctgtccctaagttagtcttgtttacctctgaattagctcactattctacaaagaaaatggctgcattcatgggtattggttccgacaattgtgtcaatatcaaaacagacgacgttggtaagatgaacattgttgacctagaaatgaagattaagatcgcaattgacaacaagtgtacccctttcatggttacagctacatcaggtactactgttttcggtgcttttgacccacttgtcgcaatctccgatttgtgtaagaagtacaacttatggctacatgtcgatgcggcatggggtggtggtgcgttaatgtcaaagaagcatagacatcttctaaacggtatcgaattagcagactccgttacctggaatccacacaaattgttagctgctcctcaacaatgttccacattccttaccagacacaagaaggtcctgtctgaaggtcattcttccaacgccaaatacttgtttcagaaggacaagttttacgacacttcttatgacactggtgacaaacatattcagtgtggtagaagagcagatgttttgaagttctggtttatgtggaaggcaaagggtacggagggtttcgagaagcacgttgataagttgtttgacaatgctaagtacttcctagatcacattaagcaaagggaaggtttccaattggtcattgcagaacctcagtgtaccaacatcatgttctggtatatcccaaagtgtctaagaggttgtgaaaatgatgcagactactatgaaagattgcataaagtcgctccaaagatcaaagaaagaatgatcaaagaaggttctatgatggtcacctaccaaccacaaggtgatttggtcaatttctttagaatcgttttccagaattctgctttagatcataaggatatggtctactttgcaaacgaattcgaaagattaggttccgacatgatcgtttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagcggccgccaacttggttcaaaccaagaaagccgtcgtgtactttgcgtattttcaacttcttcccttcaactccaaccttataacccaatccgacagttagcatacagtagaaaatccataagttaattgcaaaaaccacataattcccaatgactggcacaatttgcatgaacggcgcaaaaccaacacctatccttcttgacttgtggacaacttctccggcatcggctccagtttctggtttaggcgacactttcaaggtacccttgttgactaggttctcagcaacatactcctcctcgataactttgaccgtctttgtgccaaacacattgttcaatagtttggacgctcgcataccgaagacagactcgtccatccaatagcatgtactccgaatgctctcaatcaacggtatcaaatcgcttggtgtctttggaggcggtgttctaggtttcatcttcgccttcccgtttcctgtttcttggacttcaaaatacgggtcatatgcatcttcattggtgaacctgaacagtttgggccgagtagtgccaaacaccttcaacagtataatcttcactccttggccgattagaaacatgcttatatgtttgccggtattcttaaagtgtatactggagcccttttaatcacattttttttacttgtcagtctccatacggagtttaatgtccttatatcgatcttcatcagctccaacgggacaggaatcaataaccttccttgcctgtcccaaaaagaatgaattttttttcaaaagctttacgatgcataccacaaaaggaagattattcccacatgttccagaagtgtgcggagatacaaagggttcatgaaaacgtgaatcttctaaaaacttagcacaacaataaaaatctacaatgttacagtaagtattattttctttttgtcgacacactccaacggttagatttccaagtattcaatccaatgtattacttgtcagacagccatccactcccatcttagaacatcacttccgaaccgc

SEQ ID NO: 5 - DNA, 4743 bp, Artificial Sequence
  synthetic
cgcccagcagatatcaatgctttacttggggaaatccatcaaggtgcaaggttgaagaaggtcgacgacagcgagaaacacattgctgacggtgccgttgttggcagagtattgtaattgcaacatgttcctgtttagtctagcatatatatacttacttataaagctaaaatattgcgttttgttctagttttaactgccgtctctttcttgccgagccccctttattttcttcctttagagaggcaataaaatttcgataagcgcaatcccgaattttttctgcggcgagagagatgcgggactgggcttccgaagaacgtaaccaaactgattttatccaatataaaagagagggtgccacagctgtttgcgggagaaggtactcttatttacacggtagtcttctctcacatttcagatataatacaagcaatattcaacagcagacgcggccgcgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctccaaccaaaaggctagcatgttcggtaacatctcccaaagattggcaggtaagaacatcttgatcaccggtgcctccaccggtatcggttaccacaccgcaaagtacttcgctgaagctgccaacggtgacttgaagttggtcttggcagcaagaagaaaggaaaagttggaagcattgaaggcagacttgttggctaagtacccatccatcaaggtccacatcgaatccttggacgtctccaagaccgagaccatcgctccattcttgaagggtttgccagaagaattctccatcgttgacgttttggtcaacaacgcaggcaaggccttgggtttggacccaatcggttccgtcgacccaaaggacgtcgacgaaatgttccaaaccaacgtcttgggtatgatccaattgacccaattggtcgtccaacaaatgaaggaaagaaactccggtgacatcgtccaattgggttccgttgctggcagaaacccataccctggtggtggtatctactgtgcttccaaggctgctttgaggtccttcacccacgttttgagagaagaattgatcaacaccaagatcagagttatcgaaatcgaacctggtaacgtcgctaccgaagaattctccttgaccagattcaagggcgacaagtccaaggctgagaaggtctacgaaggtaccgaaccattgtacggtaccgacatcgccgaattgatcttgttcgctgtttccagaccacaaaacaccgtcatcgccgagaccttggtcttcgcttccaaccaggcatccgcttaccacatcttcagaggttccttggacaagtaacgatcgtaaatctaactaatgcttttactaaatatctagtacaatttttacagtccctacgtttataaatgaatttaatgaaaaaaaaatattttgtaacgatgtgtttattaagttgcgctcttccgataatcccggactttggttaatttctcaatgggtttttttttcaaaaccattgttgtagtgtaacagactttaacaaaaggacatcactctacagggcagctttaaaatccctcagtgtaattgttcttcattcataacgtggcagtcaaggactcgaggagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaaactaaaatgtttggtaatatttcccaaagacttgcaggcaagaacatcctaattacaggtgcgtccactggtatcggataccatacagcaaagtattttgcagaagctgcaaatggagacttgaagttggttttggctgcaagaagaaaggagaagctggaggcactaaaggcagacttgcttgccaagtatccatccatcaaagtccatattgagagtttggatgtctccaaaacggaaaccattgcacctttcttaaaaggtttacctgaggaattttcaattgtcgacgtgttggtcaacaatgcaggtaaggcgcttggtttggatccaattggctctgtcgatccaaaggacgtggatgaaatgttccagaccaatgttttgggtatgattcaattgacccagttggttgtacagcaaatgaaggagagaaactccggggacattgtccaactaggttcagtggctggtagaaacccatacccaggtggtggtatctactgtgcctccaaggccgcattgagatcttttacacatgtattgagagaggaattgattaataccaagattagagtgattgaaatcgagcctggaaatgttgcaactgaggaattttctttgaccagattcaaaggtgataagtccaaggccgaaaaggtctatgagggaaccgagccattgtatggtaccgatattgcagaattgattctatttgcagtttctagacctcaaaacactgttattgcagaaacacttgtttttgctagtaaccaagcttctgcttaccatattttcagaggatcattagataaattaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcgcccttacatatgataacttcgtataatgtatgctatacgaagttatcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaag

SEQ ID NO: 6 - DNA, 4370 bp, Artificial Sequence
  synthetic
ccacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagataacttcgtataatgtatgctatacgaagttatagatcgcggccgctaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacaattacaaaaagctagcatgttcggtaacatctcccaacgtttggctggtaagaacatcttgatcaccggtgctggcaccggtatcggttaccacaccgctaagtacttcgcagaagctgccaacggtgacttgaagttggttttggcagacttgagaaaggaaaagttggaagcattgaaggcagacttgttggcaaagtacccatccatcaaggttcacatcgaatccttggacgtctccaagaccgaaaccatcgctcctttcttgaagggcttgccagaggagttctccatcgtcgacgttttggtcaacaacgctggtaaggccttgggtttggacccaatcggttccgtcgacccaaaggacgtcgacgaaatgttccaaaccaacgtcttgggtatgatccaattgacccagttggtcgtccagcagatgaaggagagaaactccggtgacatcgtccaattgggttccgtcgcaggtagaaacccatacccaggtggtggtatctactgtgcttccaaggctgctttgagatccttcacccacgtcttgagagaggaattgatcaacaccaagatcagagttatcgaaatcgaaccaggtaacgtcgctaccgaagaattctccttgaccaggttcaagggtgacaagtccaaggcggagaaggtctacgagggtaccgaaccattgtacggtaccgacatcgctgaattgatcttgttcgccgtctccagacctcaaaacaccgttatcgctgaaaccttggttttcgcttccaaccaggcttccgcataccacatcttccgtggttccttggacaagcgatcgtaagcggcgaatctctggctcatgggggatatcctctttgtttggcttttttttcccattctctgttttgattatctaatgactcattgggaggattttctcacttcaagcttttttttcttgcactctttcataactccagctctctctaactgaggctacaatgccttttaacgaacttatgagacgtttctaaattatataggtatatgccaatatataattacacataaaaataaatataaataaaatataaaaataaaaataaacatcgaaaaagaagatgtgaaattgcgaagactagaaagcacaaacgagcggtctatatcggcgactcgaggctctacaagcctcatatgggttcaatgggtctgcaatgaccgcatacggacttggacaattaccttctattgaatttctgagaagagatacatctgaccagcaatgtaagcagacaatcccaattctgtaaacaacctctttgtccataattccccatcagaagagtgaaaaatgccctcaaaacgcatgcgccactcccacctctcagctgcactgcgccacctctgagggtcctttcaggggtcgactaccccggacacctcgcagaggagcgacgtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttccttgcgttttcttttcaactttttttactttttccttcaacagacaaatctaacttatatatcacatctagaatgttcggtaacatctcccaaagattggctggtaagaacatcttgatcactggcgcttccaccggtatcggttaccacaccgctaagtacttcgcggaagctgcaaacggtgacttgaagttggtcttggcagcaagaagaaaggaaaagttggaagctttgaaggcagacttgttggcaaagtacccatccatcaaggtccacatcgaatccttggacgtctccaagaccgagaccatcgcacctttcttgaagggcttgccagaagaattctccatcgttgacgtcttggtcaacaacgcaggtaaggcattgggtttggaccctatcggttccgtcgacccaaaggacgtcgacgaaatgttccaaaccaacgtcttgggtatgatccaattgacccaattggttgtccaacaaatgaaggaacgtaactccggtgacatcgttcaattgggctccgtcgctggtagaaacccataccctggtggtggtatctactgtgcatccaaggcagctttgaggtccttcacccacgtcttgagagaggagttgatcaacaccaagatcagagtcatcgaaatcgaaccaggtaacgttgctaccgaagaattctccttgaccagattcaagggcgacaagtccaaggcagaaaaggtctacgaaggtaccgaacctttgtacggtaccgacatcgccgagttgatcttgttcgcagtctccaggcctcaaaacaccgttatcgctgaaaccttggttttcgcttccaaccaggcatccgcataccacatcttcagaggttccttggacaagttaattaagtatagccatatagtttaattcctttatactttttataactatttcttacactaattattattatcaattatttattgtagaacttgactcttgcgtcgatcaccatgacagggctatcttaacaaggggtaatttttgttgatggagtcaagtagcattccgacgggaagtgtcgatgcctctgaacgaaatcttccgattagctctgcaaagaagtggaaattgtcagcgcagaattcgccgaaaaggtctatgagggaaccgagccattgtatggtaccgatattgcagaattgattctatttgcagtttctacacctcaaaacactgttattgcagaaacacttgtttttgctagtaaccaagcttctgcttaccatattttcagaggatcattagataaatagatttgatataaacgcttctataataataaataacatcaacaagtgactaaccccaagtatccaattttagacctaatgcctgaaagtgccttgcacgatttaggattcggcaccagaaagtttgtgccgtgctcgcttattggacttgtatagtgataaggcaaaaaaaaaaaacttgaaagtacttgcgtaactcagaggttgccttttcg

SEQ ID NO: 7 - DNA, 9582 bp, Artificial Sequence
  synthetic
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaattctagataaaatgtcaactgtggaagatcactcctccttacataaattgagaaaggaatctgagattctttccaatgcaaacaaaatcttagtggctaatagaggtgaaattccaattagaattttcaggtcagcccatgaattgtcaatgcatactgtggcgatctattcccatgaagatcggttgtccatgcataggttgaaggccgacgaggcttatgcaatcggtaagacgggtcaatattcgccagttcaagcttatctacaaattgacgaaattatcaaaatagcaaaggaacatgatgtttccatgatccatccaggttatggtttcttatctgaaaactccgaattcgcaaagaaggttgaagaatccggtatgatttgggttgggcctcctgctgaagttattgattctgttggtgacaaggtttctgcaagaaatttggcaattaaatgtgacgttcctgttgttcctggtaccgatggtccaattgaagacattgaacaggctaaacagtttgtggaacaatatggttatcctgtcattataaaggctgcatttggtggtggtggtagaggtatgagagttgttagagaaggtgatgatatagttgatgctttccaaagagcgtcatctgaagcaaagtctgcctttggtaatggtacttgttttattgaaagatttttggataagccaaaacatattgaggttcaattattggctgataattatggtaacacaatccatctctttgaaagagattgttctgttcaaagaagacatcaaaaggttgttgaaattgcacctgccaaaactttacctgttgaagttagaaatgctatattaaaggatgctgtaacgttagctaaaaccgctaactatagaaatgctggtactgcagaatttttagttgattcccaaaacagacattattttattgaaattaatccaagaattcaagttgaacatacaattactgaagaaatcacgggtgttgatattgttgccgctcaaattcaaattgctgcaggtgcatcattggaacaattgggtctattacaaaacaaaattacaactagaggttttgcaattcaatgtagaattacaaccgaggatcctgctaagaattttgccccagatacaggtaaaattgaggtttatagatctgcaggtggtaacggtgtcagattagatggtggtaatgggtttgccggtgctgttatatctcctcattatgactcgatgttggttaaatgttcaacatctggttctaactatgaaattgccagaagaaagatgattagagctttagttgaatttagaatcagaggtgtcaagaccaatattcctttcttattggcattgctaactcatccagttttcatttcgggtgattgttggacaacttttattgatgataccccttcgttattcgaaatggtttcttcaaagaatagagcccaaaaattattggcatatattggtgacttgtgtgtcaatggttcttcaattaaaggtcaaattggtttccctaaattgaacaaggaagcagaaatcccagatttgttggatccaaatgatgaggttattgatgtttctaaaccttctaccaatggtctaagaccgtatctattaaagtatggaccagatgcgttttccaaaaaagttcgtgaattcgatggttgtatgattatggataccacctggagagatgcacatcaatcattattggctacaagagttagaactattgatttactgagaattgctccaacgactagtcatgccttacaaaatgcatttgcattagaatgttggggtggcgcaacatttgatgttgcgatgaggttcctctatgaagatccttgggagagattaagacaacttagaaaggcagttccaaatattcctttccaaatgttattgagaggtgctaatggtgttgcttattcgtcattacctgataatgcaattgatcattttgttaagcaagcaaaggataatggtgttgatattttcagagtctttgatgctttgaacgatttggaacaattgaaggttggtgttgatgctgtcaagaaagccggaggtgttgttgaagctacagtttgttactcaggtgatatgttaattccaggtaaaaagtataacttggattattatttagagactgttggaaagattgtggaaatgggtacccatattttaggtattaaggatatggctggcacgttaaagccaaaggctgctaagttgttgattggctcgatcagatcaaaataccctgacttggttatccatgtccatacccatgactctgctggtaccggtatttcaacttatgttgcatgcgcattggcaggtgccgacattgtcgattgtgcaatcaattcgatgtctggtttaacctctcaaccttcaatgagtgcttttattgctgctttagatggtgatatcgaaactggtgttccagaacattttgcaagacaattagatgcatactgggcagaaatgagattgttatactcatgtttcgaagccgacttgaagggaccagacccagaagtttataaacatgaaattccaggtggacagttgactaacctaatcttccaagcccaacaagttggtttgggtgaacaatgggaagaaactaagaagaagtatgaagatgctaacatgttgttgggtgatattgtcaaggttaccccaacctccaaggttgttggtgatttagcccaatttatggtttctaataaattagaaaaagaagatgttgaaaaacttgctaatgaattagatttcccagattcagttcttgatttctttgaaggattaatgggtacaccatatggtggattcccagagcctttgagaacaaatgtcatttccggcaagagaagaaaattaaagggtagaccaggtttagaattagaacctttcaacctcgaggaaatcagagaaaatttggtttccagatttggtccaggtattactgaatgtgatgttgcatcttataacatgtatccaaaggtttacgagcaatatcgtaaggtggttgaaaaatatggtgatttatctgttttaccaacaaaagcatttttggctcctccaactattggtgaagaagttcatgtggaaattgagcaaggtaagactttgattattaagttattagccatttctgacttgtctaaatctcatggtacaagagaagtatactttgaattgaatggtgaaatgagaaaggttacaattgaagataaaacagctgcaattgagactgttacaagagcaaaggctgacggacacaatccaaatgaagttggtgcgccaatggctggtgtcgttgttgaagttagagtgaagcatggaacagaagttaagaagggtgatccattagccgttttgagtgcaatgaaaatggaaatggttatttctgctcctgttagtggtagggtcggtgaagtttttgtcaacgaaggcgattccgttgatatgggtgatttgcttgtgaaaattgccaaagatgaagcgccagcagcttaattaattctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaaggtttaaacgattggtagttctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagaggtctcttgataatgtttcattaccacgtggcatccccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctctcgcgataaaatgtccagaggcttctttactgagaacattacgcaattgccaccagaccctttgtttggtcttaaggccaggttcagcaatgactcacgtgaaaacaaggtcgatttaggtattggggcatatagggacgacaacggtaagccatggatcttaccatctgtcaggttggccgaaaacttgattcagaactccccagactacaaccatgagtacctaccaatcggtggacttgctgatttcacttctgctgcggcaagagttgtatttggaggcgattctaaagccatttcgcaaaaccgtcttgtctccatccagagtttgtcaggtacaggtgccttacatgttgctggtctatttatcaagcgccaatacaagtctcttgatggcacttccgaagaccctctaatatatctatcggaacctacatgggccaaccacgttcaaatctttgaagttattggtctcaagcctgtattctatccatattggcatgccgcaagcaagaccttggatctgaagggctacttaaaggcaatcaacgatgctccagaagggtcggtttttgtattgcatgcaacggctcataaccctactggtttggatccaacacaagaacaatggatggagattttggccgctataagtgccaaaaagcatctgccattatttgattgtgcatatcagggtttcacctccgggtctctagatagagatgcttgggctgttcgggaagctgtcaacaatgacaagtacgaattcccgggaattattgtctgtcaatcgtttgcgaaaaatgttggcatgtatggtgaacggattggtgcagttcatattgttctacctgaatcagacgcttccctaaacagcgccatcttctcccaattgcaaaagacaatcagatcggagatttccaatccaccaggatacggtgcaaagattgtgtctaaagttttgaacactccggaactttacaaacagtgggagcaagatttgatcaccatgtcttcgagaatcactgcaatgagaaaggagctagtaaatgagctcgagcgtcttggaacccctggcacttggagacacatcaccgagcaacagggtatgttttcctttactggtttgaacccggagcaggttgccaagctagagaaggagcatggtgtttatcttgttcgtagtggacgtgcaagtattgcaggcctcaacatggggaacgtcaagtatgttgccaaggccattgactctgtcgtgagagacctttaggggcccttaatttattttactagtttatttttgctcctgagaataggattacaaacacttaaagtctttaattacaactatatataatattctgttggttttcttgaattggttcgctgcgattcatgcctcccattcaccaaaggtggagtgggaaataacggttttactgcggtaattagcagaggcaagaacaggatacactttttgatgataaatctgtattatagtcgagcctatttaggaaatcaaattttcttgtgtttacttttcaaataaataatgttcgaaaatttttactttactccttcatttaactataccagacgttatatcatcaacaccttctgaccatatacagctcaagatgtttaagagtctgttaaattttttcaatccatttcatggagtaccaggaggtgctacaaaagagaattcgcccttacatatggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatggaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgcccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaacttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttc

SEQ ID NO: 8 - DNA, 7074 bp, Artificial Sequence
  synthetic
cctattttctattctctatgtaactggggtcaggatttgacattttactggggctctggtatcgccaattcttggagaatgagcggagatattactgctgagttcacccgcccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaacttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattgcatactagttcataacttcgtataatgtatgctatacgaagttattaattaacaagggcgatttctgcagatatcggccttcaacgcgtttcctcgacatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacatgctagctaaaatgagagttgattccaagatcattgtcaagaaagagactcaagacgaaaacttgtatcaatccttggccgaaagatccaagcacgaggagtttcttagaaaggcagtcgatctgttggttgagagagttgtttttggtagatctactagatcttctaaggtcgttgaatgggctgcaccagacgaaatcaagaaagcaattgatttgaagcctagattaggtccagcttcccatgatgaactcttggcgttcatggcaaatgtcgctagatattctgttaacactggtcatccatacttcgttaaccaacttttctcttcagttgatccatatggtttggtcggtcaatggttaaccgacgccttaaacccatccgtttacaccttcgaagtcgcacctgtcttcactttgatggaagaagaagttttgagggaaatgagaaagatcgtcggttggccagaaggtgaaggtgatggcattttctgtccaggtggttccatcgcaaatggttacgcaatttcttgtgcaagacaccacttttacccagaagttaagtacaaaggtgttcacgctgttcctaagttggttttgttcacgtccgagcttgctcactattccaccaaaaagatggcagcattcatgggtattggctccgacaattgtgtcaatatcaaaaccgatgatgttggtaagatgaacatcgttgatttggaaatgaagatcaagatcgcaattgataacaagtgtactccattcatggttaccgcaaccagtggtaccactgtctttggtgcattcgaccctttggttgcaattagtgacttatgtaagaagtataacttatggttgcacgttgacgctgcttggggtggtggtgccttgatgtctaagaagcatagacacttgttgaatggcattgaattggcagattctgttacttggaatccacataagttgcttgccgctccacaacaatgttctaccttcttaaccagacataagaaagtcttatctgaaggtcacagttccaatgccaagtatctgtttcaaaaggataagttctacgatacatcttacgacactggcgacaaacatattcaatgtggtagaagagcagacgtcttgaagttttggttcatgtggaaggcaaaaggtacagaaggtttcgaaaagcatgttgacaagctattcgacaacgcgaagtactttttggatcacatcaaacaaagagaaggttttcagctagttatcgctgaaccacaatgtaccaacattatgttctggtacattccaaagtgtctccgtggttgtgaaaacgatgcagattactatgagagactccacaaggtcgcacctaagatcaaagaacgtatgatcaaagaaggttctatgatggtcacctaccaaccacaaggtgacttagttaacttctttagaatcgttttccaaaactccgcattagatcacaaggatatggtttactttgcaaatgagttcgaaagattaggttccgacatgattgtttaaggcgcgccatctaatagtttaatcacagcttatagtctattatagttttcttttttaaacattgttgtattttgtccccccccctctaattgatgatgattatcctataagaatccaataaaacgatggaaactaatactttctcctttgtcatgtggtctttagtatttcttgaacattggctctgatttctcgactttatagtcctattaaaatctctgttagttctcgatcgttgtatctcgtttcttgtctctttggtggatgattttgcgtgcgaacatgtttttttccctttctctcaccatcatcgtgtagttcttgtcaccatccccccaccccttccttctctcattgattctataagagcttatccacagaggtgcagtaacgaggtagtttaaccttcgagtggatcaaaatgtcacacaggaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacatctagataaaatgtctattagtgaaaaatattttcctcaagaacctcaatctcctgaattgaaaactgcaattccaggtcctcaatcaaaggcaaagctcgaggaattatctgctgtctatgatacaaaggctgcatattttgttaccgactactacaaatctcttggtaactatattgtggatgcagatggcaacaagctactagattcttattgccaaatctcttctatcgcattgggttacaataatccagcattattaaaagtagcacattctgatgaaatggcagttgctttatgtaacagacctgctttggcatgttttccatccactgattactatgaaatactaaagaagggattgttgtccgttgctccaaagggattagataaggtttgtactgcacacacgggatctgatgccaatgaaatggcatttaaggctgcatttttgtttcaagcaagtaagaagagaggtgacaaaccatttaccagcgaagagctggaatctgtcatggagaacaagttgccaggcacctctgacatggttatcctgtcatttgaaaaagggttccatggtagattgtttggatctttatctaccactagatctaaagctattcacaaactggatattcctgcgtttgaatggccaaaggctccattccctcagttaaagtatcctctggatcaattccaagctgaaaacaaagcagaagaagaaagatgtttgaaggctttagaggaaattattgtcaactctcctgccaaaattgcagctgcaatcattgaaccggtccaatctgaaggtggtgataatcatgcttcaccagaattcttccaaggtattagagaaatcaccaaaaagcacggtgtcattcttattgttgatgaagttcaaacaggaggtggtgcttctggtaagatgtggttacatgaacactatggcattgtcccagacatcatgactttttctaaaaaaatgcaaaatgcaggtttctttttcagtgaagcaggtcttgctggggaccaaccattcagacaattcaatacctggtgcggtgatccatcaaaagctctaattgcaagaaccataattgaagaaattaaagataagaacctattgactagtgttaccgaaacaggtgactacctatattcaaagctcgaagcaatttcagcaaagtatgacaaaatgatcaacttgagaggtaagggaagaggtttctttattgcatttgatgccccaacaccggagttaagaaacaaatttattgctgaatgtaagaaattaggtttaaacattggtggatgcggtgaacaaggtgttagattgagacctgcattagtttttgaaaagaagcatgctgatatcttagcctccattattgatcaagctttttccaaaatttaaaagttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccgagcttggataggtcactctctattttcgtttctcccttccctgatagaggggtgatatgtaattaagaataatatataattttataataaaaactaaaacaatccatcaatctcaccatcttcgttgacttcaacattcataaatccggcataagttgatagacctggaattgtcatgatctttgcagctagtgcatataaatatcctgctcctgcacttattctaacttctctgattgggaagatgaaatcctttggaacacctttcaatgttggatcatgggagagagaatattgcgtctttgcaatacaaacaggcagcttgccaaaaccttgactctcatactcttcaatctgtttcttggccaactcagatagttcaatatcctttgctccatacatcttggtggcaattgttcttagtctatcttctaacgaaccatctaatgaataaagatagttaggaggttgactttcttccaacggtctttcctcggtagcacgtacaattgcctcagctaacttgattgcacccttaccaccttgtgaccaatggtctgactcaacagcatcaaatgcaccagccttgatggcttcttctctaatggtctcaatttccttctcggtgtcggtagcaaatttgttaattgcaacaacaactggggccccataactcttggcattgc

SEQ ID NO: 9 - DNA, 9001 bp, Artificial Sequence
  synthetic
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaattctagataaaatgtcaactgtggaagatcactcctccttacataaattgagaaaggaatctgagattctttccaatgcaaacaaaatcttagtggctaatagaggtgaaattccaattagaattttcaggtcagcccatgaattgtcaatgcatactgtggcgatctattcccatgaagatcggttgtccatgcataggttgaaggccgacgaggcttatgcaatcggtaagacgggtcaatattcgccagttcaagcttatctacaaattgacgaaattatcaaaatagcaaaggaacatgatgtttccatgatccatccaggttatggtttcttatctgaaaactccgaattcgcaaagaaggttgaagaatccggtatgatttgggttgggcctcctgctgaagttattgattctgttggtgacaaggtttctgcaagaaatttggcaattaaatgtgacgttcctgttgttcctggtaccgatggtccaattgaagacattgaacaggctaaacagtttgtggaacaatatggttatcctgtcattataaaggctgcatttggtggtggtggtagaggtatgagagttgttagagaaggtgatgatatagttgatgctttccaaagagcgtcatctgaagcaaagtctgcctttggtaatggtacttgttttattgaaagatttttggataagccaaaacatattgaggttcaattattggctgataattatggtaacacaatccatctctttgaaagagattgttctgttcaaagaagacatcaaaaggttgttgaaattgcacctgccaaaactttacctgttgaagttagaaatgctatattaaaggatgctgtaacgttagctaaaaccgctaactatagaaatgctggtactgcagaatttttagttgattcccaaaacagacattattttattgaaattaatccaagaattcaagttgaacatacaattactgaagaaatcacgggtgttgatattgttgccgctcaaattcaaattgctgcaggtgcatcattggaacaattgggtctattacaaaacaaaattacaactagaggttttgcaattcaatgtagaattacaaccgaggatcctgctaagaattttgccccagatacaggtaaaattgaggtttatagatctgcaggtggtaacggtgtcagattagatggtggtaatgggtttgccggtgctgttatatctcctcattatgactcgatgttggttaaatgttcaacatctggttctaactatgaaattgccagaagaaagatgattagagctttagttgaatttagaatcagaggtgtcaagaccaatattcctttcttattggcattgctaactcatccagttttcatttcgggtgattgttggacaacttttattgatgataccccttcgttattcgaaatggtttcttcaaagaatagagcccaaaaattattggcatatattggtgacttgtgtgtcaatggttcttcaattaaaggtcaaattggtttccctaaattgaacaaggaagcagaaatcccagatttgttggatccaaatgatgaggttattgatgtttctaaaccttctaccaatggtctaagaccgtatctattaaagtatggaccagatgcgttttccaaaaaagttcgtgaattcgatggttgtatgattatggataccacctggagagatgcacatcaatcattattggctacaagagttagaactattgatttactgagaattgctccaacgactagtcatgccttacaaaatgcatttgcattagaatgttggggtggcgcaacatttgatgttgcgatgaggttcctctatgaagatccttgggagagattaagacaacttagaaaggcagttccaaatattcctttccaaatgttattgagaggtgctaatggtgttgcttattcgtcattacctgataatgcaattgatcattttgttaagcaagcaaaggataatggtgttgatattttcagagtctttgatgctttgaacgatttggaacaattgaaggttggtgttgatgctgtcaagaaagccggaggtgttgttgaagctacagtttgttactcaggtgatatgttaattccaggtaaaaagtataacttggattattatttagagactgttggaaagattgtggaaatgggtacccatattttaggtattaaggatatggctggcacgttaaagccaaaggctgctaagttgttgattggctcgatcagatcaaaataccctgacttggttatccatgtccatacccatgactctgctggtaccggtatttcaacttatgttgcatgcgcattggcaggtgccgacattgtcgattgtgcaatcaattcgatgtctggtttaacctctcaaccttcaatgagtgcttttattgctgctttagatggtgatatcgaaactggtgttccagaacattttgcaagacaattagatgcatactgggcagaaatgagattgttatactcatgtttcgaagccgacttgaagggaccagacccagaagtttataaacatgaaattccaggtggacagttgactaacctaatcttccaagcccaacaagttggtttgggtgaacaatgggaagaaactaagaagaagtatgaagatgctaacatgttgttgggtgatattgtcaaggttaccccaacctccaaggttgttggtgatttagcccaatttatggtttctaataaattagaaaaagaagatgttgaaaaacttgctaatgaattagatttcccagattcagttcttgatttctttgaaggattaatgggtacaccatatggtggattcccagagcctttgagaacaaatgtcatttccggcaagagaagaaaattaaagggtagaccaggtttagaattagaacctttcaacctcgaggaaatcagagaaaatttggtttccagatttggtccaggtattactgaatgtgatgttgcatcttataacatgtatccaaaggtttacgagcaatatcgtaaggtggttgaaaaatatggtgatttatctgttttaccaacaaaagcatttttggctcctccaactattggtgaagaagttcatgtggaaattgagcaaggtaagactttgattattaagttattagccatttctgacttgtctaaatctcatggtacaagagaagtatactttgaattgaatggtgaaatgagaaaggttacaattgaagataaaacagctgcaattgagactgttacaagagcaaaggctgacggacacaatccaaatgaagttggtgcgccaatggctggtgtcgttgttgaagttagagtgaagcatggaacagaagttaagaagggtgatccattagccgttttgagtgcaatgaaaatggaaatggttatttctgctcctgttagtggtagggtcggtgaagtttttgtcaacgaaggcgattccgttgatatgggtgatttgcttgtgaaaattgccaaagatgaagcgccagcagcttaattaattctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaaggtttaaacgattggtagttctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagaggtctcttgataatgtttcattaccacgtggcatccccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctctcgcgataaaatgtccagaggcttctttactgagaacattacgcaattgccaccagaccctttgtttggtcttaaggccaggttcagcaatgactcacgtgaaaacaaggtcgatttaggtattggggcatatagggacgacaacggtaagccatggatcttaccatctgtcaggttggccgaaaacttgattcagaactccccagactacaaccatgagtacctaccaatcggtggacttgctgatttcacttctgctgcggcaagagttgtatttggaggcgattctaaagccatttcgcaaaaccgtcttgtctccatccagagtttgtcaggtacaggtgccttacatgttgctggtctatttatcaagcgccaatacaagtctcttgatggcacttccgaagaccctctaatatatctatcggaacctacatgggccaaccacgttcaaatctttgaagttattggtctcaagcctgtattctatccatattggcatgccgcaagcaagaccttggatctgaagggctacttaaaggcaatcaacgatgctccagaagggtcggtttttgtattgcatgcaacggctcataaccctactggtttggatccaacacaagaacaatggatggagattttggccgctataagtgccaaaaagcatctgccattatttgattgtgcatatcagggtttcacctccgggtctctagatagagatgcttgggctgttcgggaagctgtcaacaatgacaagtacgaattcccgggaattattgtctgtcaatcgtttgcgaaaaatgttggcatgtatggtgaacggattggtgcagttcatattgttctacctgaatcagacgcttccctaaacagcgccatcttctcccaattgcaaaagacaatcagatcggagatttccaatccaccaggatacggtgcaaagattgtgtctaaagttttgaacactccggaactttacaaacagtgggagcaagatttgatcaccatgtcttcgagaatcactgcaatgagaaaggagctagtaaatgagctcgagcgtcttggaacccctggcacttggagacacatcaccgagcaacagggtatgttttcctttactggtttgaacccggagcaggttgccaagctagagaaggagcatggtgtttatcttgttcgtagtggacgtgcaagtattgcaggcctcaacatggggaacgtcaagtatgttgccaaggccattgactctgtcgtgagagacctttaggggcccttaatttattttactagtttatttttgctcctgagaataggattacaaacacttaaagtctttaattacaactatatataatattctgttggttttcttgaattggttcgctgcgattcatgcctcccattcaccaaaggtggagtgggaaataacggttttactgcggtaattagcagaggcaagaacaggatacactttttgatgataaatctgtattatagtcgagcctatttaggaaatcaaattttcttgtgtttacttttcaaataaataatgttcgaaaatttttactttactccttcatttaactataccagacgttatatcatcaacaccttctgaccatatacagctcaagatgtttaagagtctgttaaattttttcaatccatttcatggagtaccaggaggtgctacaaaagagaattcgcccttacatatgataacttcgtataatgtatgctatacgaagttatcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagataacttcgtataatgtatgctatacgaagttatagatccgc

SEQ ID NO: 10 - DNA, 6608 bp, Artificial Sequence
  synthetic
gggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagataacttcgtataatgtatgctatacgaagttatagatccgcgaagggcgaattctgcagatatccatcacactggcggccgcttaacatcttcaacgcgtttcctcgacatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacatgctagctaaaatgagagttgattccaagatcattgtcaagaaagagactcaagacgaaaacttgtatcaatccttggccgaaagatccaagcacgaggagtttcttagaaaggcagtcgatctgttggttgagagagttgtttttggtagatctactagatcttctaaggtcgttgaatgggctgcaccagacgaaatcaagaaagcaattgatttgaagcctagattaggtccagcttcccatgatgaactcttggcgttcatggcaaatgtcgctagatattctgttaacactggtcatccatacttcgttaaccaacttttctcttcagttgatccatatggtttggtcggtcaatggttaaccgacgccttaaacccatccgtttacaccttcgaagtcgcacctgtcttcactttgatggaagaagaagttttgagggaaatgagaaagatcgtcggttggccagaaggtgaaggtgatggcattttctgtccaggtggttccatcgcaaatggttacgcaatttcttgtgcaagacaccacttttacccagaagttaagtacaaaggtgttcacgctgttcctaagttggttttgttcacgtccgagcttgctcactattccaccaaaaagatggcagcattcatgggtattggctccgacaattgtgtcaatatcaaaaccgatgatgttggtaagatgaacatcgttgatttggaaatgaagatcaagatcgcaattgataacaagtgtactccattcatggttaccgcaaccagtggtaccactgtctttggtgcattcgaccctttggttgcaattagtgacttatgtaagaagtataacttatggttgcacgttgacgctgcttggggtggtggtgccttgatgtctaagaagcatagacacttgttgaatggcattgaattggcagattctgttacttggaatccacataagttgcttgccgctccacaacaatgttctaccttcttaaccagacataagaaagtcttatctgaaggtcacagttccaatgccaagtatctgtttcaaaaggataagttctacgatacatcttacgacactggcgacaaacatattcaatgtggtagaagagcagacgtcttgaagttttggttcatgtggaaggcaaaaggtacagaaggtttcgaaaagcatgttgacaagctattcgacaacgcgaagtactttttggatcacatcaaacaaagagaaggttttcagctagttatcgctgaaccacaatgtaccaacattatgttctggtacattccaaagtgtctccgtggttgtgaaaacgatgcagattactatgagagactccacaaggtcgcacctaagatcaaagaacgtatgatcaaagaaggttctatgatggtcacctaccaaccacaaggtgacttagttaacttctttagaatcgttttccaaaactccgcattagatcacaaggatatggtttactttgcaaatgagttcgaaagattaggttccgacatgattgtttaaggcgcgccatctaatagtttaatcacagcttatagtctattatagttttcttttttaaacattgttgtattttgtccccccccctctaattgatgatgattatcctataagaatccaataaaacgatggaaactaatactttctcctttgtcatgtggtctttagtatttcttgaacattggctctgatttctcgactttatagtcctattaaaatctctgttagttctcgatcgttgtatctcgtttcttgtctctttggtggatgattttgcgtgcgaacatgtttttttccctttctctcaccatcatcgtgtagttcttgtcaccatccccccaccccttccttctctcattgattctataagagcttatccacagaggtgcagtaacgaggtagtttaaccttcgagtggatcaaaatgtcacacaggaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacatctagataaaatgtctattagtgaaaaatattttcctcaagaacctcaatctcctgaattgaaaactgcaattccaggtcctcaatcaaaggcaaagctcgaggaattatctgctgtctatgatacaaaggctgcatattttgttaccgactactacaaatctcttggtaactatattgtggatgcagatggcaacaagctactagattcttattgccaaatctcttctatcgcattgggttacaataatccagcattattaaaagtagcacattctgatgaaatggcagttgctttatgtaacagacctgctttggcatgttttccatccactgattactatgaaatactaaagaagggattgttgtccgttgctccaaagggattagataaggtttgtactgcacacacgggatctgatgccaatgaaatggcatttaaggctgcatttttgtttcaagcaagtaagaagagaggtgacaaaccatttaccagcgaagagctggaatctgtcatggagaacaagttgccaggcacctctgacatggttatcctgtcatttgaaaaagggttccatggtagattgtttggatctttatctaccactagatctaaagctattcacaaactggatattcctgcgtttgaatggccaaaggctccattccctcagttaaagtatcctctggatcaattccaagctgaaaacaaagcagaagaagaaagatgtttgaaggctttagaggaaattattgtcaactctcctgccaaaattgcagctgcaatcattgaaccggtccaatctgaaggtggtgataatcatgcttcaccagaattcttccaaggtattagagaaatcaccaaaaagcacggtgtcattcttattgttgatgaagttcaaacaggaggtggtgcttctggtaagatgtggttacatgaacactatggcattgtcccagacatcatgactttttctaaaaaaatgcaaaatgcaggtttctttttcagtgaagcaggtcttgctggggaccaaccattcagacaattcaatacctggtgcggtgatccatcaaaagctctaattgcaagaaccataattgaagaaattaaagataagaacctattgactagtgttaccgaaacaggtgactacctatattcaaagctcgaagcaatttcagcaaagtatgacaaaatgatcaacttgagaggtaagggaagaggtttctttattgcatttgatgccccaacaccggagttaagaaacaaatttattgctgaatgtaagaaattaggtttaaacattggtggatgcggtgaacaaggtgttagattgagacctgcattagtttttgaaaagaagcatgctgatatcttagcctccattattgatcaagctttttccaaaatttaaaagttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccgagcttggataggtcactctctattttcgtttctcccttccctgatagaggggtgatatgtaattaagaataatatataattttataataaaaactaaaacaatccatcaatctcaccatcttcgttgacttcaacattcataaatccggcataagttgatagacctggaattgtcatgatctttgcagctagtgcatataaatatcctgctcctgcacttattctaacttctctgattgggaagatgaaatcctttggaacacctttcaatgttggatcatgggagagagaatattgcgtctttgcaatacaaacaggcagcttgccaaaaccttgactctcatactcttcaatctgtttcttggccaactcagatagttcaatatcctttgctccatacatcttggtggcaattgttcttagtctatcttctaacgaaccatctaatgaataaagatagttaggaggttgactttcttccaacggtctttcctcggtagcacgtacaattgcctcagctaacttgattgcacccttaccaccttgtgaccaatggtctgactcaacagcatcaaatgcaccagccttga

SEQ ID NO: 11 - DNA, 5579 bp, Artificial Sequence
  synthetic
ggccgcatcgaccagcgccagcaccaccagcgccatctttgattccttcaatcacaggtggcccagcagatatcaatgctttacttggggaaatccatcaaggtgcaaggttgaagaaggtcgacgacagcgagaaacacattgctgacggtgccgttgttggcagagtattgtaattgcaacatgttcctgtttagtctagcatatatatacttacttataaagctaaaatattgcgttttgttctagttttaactgctgtctctttcttgccgagccccctttattttcttcctttagagaggcaataatatttcgataagcgcaatcccgaattttttctgcggcgagagagatgcgggactgggcttccgaagaacgtaaccaaactgattttatccaatataaaagagagggtgccacagctgtttgcaggagaaggtactcttatttacacggtagtctcctctcacatttcagatataatacaagcaatattcaacagcagacgtcacagtatcggcatctctgtcacaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacaattacaaaaaaatgttcggtaacatctcccaacgtttggctggtaagaacatcttgatcaccggtgctggcaccggtatcggttaccacaccgctaagtacttcgcagaagctgccaacggtgacttgaagttggttttggcagacttgagaaaggaaaagttggaagcattgaaggcagacttgttggcaaagtacccatccatcaaggttcacatcgaatccttggacgtctccaagaccgaaaccatcgctcctttcttgaagggcttgccagaggagttctccatcgtcgacgttttggtcaacaacgctggtaaggccttgggtttggacccaatcggttccgtcgacccaaaggacgtcgacgaaatgttccaaaccaacgtcttgggtatgatccaattgacccagttggtcgtccagcagatgaaggagagaaactccggtgacatcgtccaattgggttccgtcgcaggtagaaacccatacccaggtggtggtatctactgtgcttccaaggctgctttgagatccttcacccacgtcttgagagaggaattgatcaacaccaagatcagagttatcgaaatcgaaccaggtaacgtcgctaccgaagaattctccttgaccaggttcaagggtgacaagtccaaggcggagaaggtctacgagggtaccgaaccattgtacggtaccgacatcgctgaattgatcttgttcgccgtctccagacctcaaaacaccgttatcgctgaaaccttggttttcgcttccaaccaggcttccgcataccacatcttccgtggttccttggacaagtaacgcggcgaatctctggctcatgggggatatcctctttgtttggcttttttttcccattctctgttttgattatctaatgactcattgggaggattttctcacttcaagcttttttttcttgcactctttcataactccagctctctctaactgaggctacaatgccttttaacgaacttatgagatgtttctaaattatataggtatatgccaatatataattacacataaaaataaatataaataaaatataaaaataaaaataaacatcgaaaaagaagatgtgaaattgcgaagattagaaagcacaaacgagcggtctatatcggcgagcttttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgccaatacattttgcgccgccacctttcaggttgtgtcactccgaaggaccatattgggtaatcgtgcaatttctggaagagagtccgcgagaagtgaggcccccactgtaaatcctcgagggggcatggagtatggggcatggaggatggaggatgggggtgggggggggggaaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattacacacgttatactaacacaacaaaaacaaaaacaacgacaacaacaacaactataatgtttggtaatatttcccaaagacttgcaggcaagaacatcctaattacaggtgcgtccactggtatcggataccatacagcaaagtattttgcagaagctgcaaatggagacttgaagttggttttggctgcaagaagaaaggagaagctggaggcactaaaggcagacttgcttgccaagtatccatccatcaaagtccatattgagagtttggatgtctccaaaacggaaaccattgcacctttcttaaaaggtttacctgaggaattttcaattgtcgacgtgttggtcaacaatgcaggtaaggcgcttggtttggatccaattggctctgtcgatccaaaggacgtggatgaaatgttccagaccaatgttttgggtatgattcaattgacccagttggttgtacagcaaatgaaggagagaaactccggggacattgtccaactaggttcagtggctggtagaaacccatacccaggtggtggtatctactgtgcctccaaggccgcattgagatcttttacacatgtattgagagaggaattgattaataccaagattagagtgattgaaatcgagcctggaaatgttgcaactgaggaattttctttgaccagattcaaaggtgataagtccaaggccgaaaaggtctatgagggaaccgagccattgtatggtaccgatattgcagaattgattctatttgcagtttctagacctcaaaacactgttattgcagaaacacttgtttttgctagtaaccaagcttctgcttaccatattttcagaggatcattagataaatagaggaatcaaacatagatcaacgtaatgaatttaatataaaatacacttatagcttccacatagtagtatatagtttaacgaattaaaaaaataaaaataaagactagagaaggtgtaaaaaaacggcttttatccaagccttccttttgttgttgtatagaagtactgctacttagagatttattattattattattattattattattattattattatcataagtccgtctcagcgagctcttctagaataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgtaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaatctagatgaaaaagcctgaactcaccgcgacgtctgtcgagaagtttctgatcgaaaagttcgacagcgtatccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcaacgc

SEQ ID NO: 12 - DNA, 5364 bp, Artificial Sequence
  synthetic
ggccgcatcggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagaaattcggatccggtagatacattgatgctatcaatccagagaactggaaagattgtgtagccttgaaaaacggtgaaacttacgggtccaagattgtctacagattttcctgatttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccataacttcgtataatgtatgctatacgaagttatgtcacagtatcggcatctctgtcatctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagagatctcttgataatgtttcattaccacgtggcatccccccccacccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacatctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctccaaccaaacaaatgtttggtaatatttcccaaagacttgcaggcaagaacatcctaattacaggtgcgtccactggtatcggataccatacagcaaagtattttgcagaagctgcaaatggagacttgaagttggttttggctgcaagaagaaaggagaagctggaggcactaaaggcagacttgcttgccaagtatccatccatcaaagtccatattgagagtttggatgtctccaaaacggaaaccattgcacctttcttaaaaggtttacctgaggaattttcaattgtcgacgtgttggtcaacaatgcaggtaaggcgcttggtttggatccaattggctctgtcgatccaaaggacgtggatgaaatgttccagaccaatgttttgggtatgattcaattgacccagttggttgtacagcaaatgaaggagagaaactccggggacattgtccaactaggttcagtggctggtagaaacccatacccaggtggtggtatctactgtgcctccaaggccgcattgagatcttttacacatgtattgagagaggaattgattaataccaagattagagtgattgaaatcgagcctggaaatgttgcaactgaggaattttctttgaccagattcaaaggtgataagtccaaggccgaaaaggtctatgagggaaccgagccattgtatggtaccgatattgcagaattgattctatttgcagtttctagacctcaaaacactgttattgcagaaacacttgtttttgctagtaaccaagcttctgcttaccatattttcagaggatcattagataaatagtaactaaatctaactaatgcttttactaaatatctagtacaatttttacagtccctacgtttataaatgaatttaatgaaaaaaaaatattttgtaacgatgtgtttattaagttgcgttcttccgataatcccggactttggttaatttctcaatgggtttttttttcaaaaccattgttgtagtgtaacagactttaacaaaaggacatcactctacagggcagctttaaaatccctcagtgtaattgttcttcattcataacgtggcagtcaaggagcttaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacatctcggttagcgtatcccccttcctcggcttttacaaatgatgcgctcttaatagtgtgtcgttatccttttggcattgacgggggagggaaattgattgagcgcatccatattttggcggactgctgaggacaatggtggtttttccgggtggcgtgggctacaaatgatacgatggtttttttcttttcggagaaggcgtataaaaaggacacggagaacccatttattctaataacagttgagcttctttaattatttgttaatataatattctattattatatattttcttcccaataaaacaaaataaaacaaaacacagcaaaacacaaaaattataatgtttggtaatatttcccaaagacttgcaggcaagaacatcctaattacaggtgcgtccactggtatcggataccatacagcaaagtattttgcagaagctgcaaatggagacttgaagttggttttggctgcaagaagaaaggagaagctggaggcactaaaggcagacttgcttgccaagtatccatccatcaaagtccatattgagagtttggatgtctccaaaacggaaaccattgcacctttcttaaaaggtttacctgaggaattttcaattgtcgacgtgttggtcaacaatgcaggtaaggcgcttggtttggatccaattggctctgtcgatccaaaggacgtggatgaaatgttccagaccaatgttttgggtatgattcaattgacccagttggttgtacagcaaatgaaggagagaaactccggggacattgtccaactaggttcagtggctggtagaaacccatacccaggtggtggtatctactgtgcctccaaggccgcattgagatcttttacacatgtattgagagaggaattgattaataccaagattagagtgattgaaatcgagcctggaaatgttgcaactgaggaattttctttgaccagattcaaaggtgataagtccaaggccgaaaaggtctatgagggaaccgagccattgtatggtaccgatattgcagaattgattctatttgcagtttctagacctcaaaacactgttattgcagaaacacttgtttttgctagtaaccaagcttctgcttaccatattttcagaggatcattagataaatagaggaccgcttgatcacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccgagcttggataggtcactctctattttcgtttctcccttccctgatagagtccgtctcagcgagctcttctagaatttgatataaacgcttctataataataaataacatcaacaagtgactaaccccaagtatccaattttagatctaatgcctgaaagtgccttgcacgatttaggattcggcaccagaaagtttgtgccgtgctcgcttattggacttgtatagtgataaggcaaaaaaaaaaaacttgaaagtacttgcgtaactcagaggttgccttttcttatcttggagtcaaaagagattcttttaattaacaaatgaagaacacagtaccgctattaattgcttctgcagtgtgtgtcactgccaacattatcccagaaggtttccatgttagggaaaggttgcaaaatgttttaggcaataaccattatcatccaattcttgatgagtatcttcaaagctccacagagccatctgactttggatctttaacaccagaagtttcggaggcatgggaatatttggagaagacattcgataggtcggttatagaagatgccattaagcaacgc

SEQ ID NO: 13 - DNA, 9580 bp, Artificial Sequence
  synthetic
ggccgcatcgaccagcgccagcaccaccagcgccatctttgattccttcaatcacaggtggcccagcagatatcaatgctttacttggggaaatccatcaaggtgcaaggttgaagaaggtcgacgacagcgagaaacacattgctgacggtgccgttgttggcagagtattgtaattgcaacatgttcctgtttagtctagcatatatatacttacttataaagctaaaatattgcgttttgttctagttttaactgctgtctctttcttgccgagccccctttattttcttcctttagagaggcaataatatttcgataagcgcaatcccgaattttttctgcggcgagagagatgcgggactgggcttccgaagaacgtaaccaaactgattttatccaatataaaagagagggtgccacagctgtttgcaggagaaggtactcttatttacacggtagtctcctctcacatttcagatataatacaagcaatattcaacagcagacgtcacagtatcggcatctctgtcatctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagagatctcttgataatgtttcattaccacgtggcatcccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacatctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctccaaccaaacaaatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaactctgatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattggccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaactctggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataataactaaatctaactaatgcttttactaaatatctagtacaatttttacagtccctacgtttataaatgaatttaatgaaaaaaaaatattttgtaacgatgtgtttattaagttgcgttcttccgataatcccggactttggttaatttctcaatgggtttttttttcaaaaccattgttgtagtgtaacagactttaacaaaaggacatcactctacagggcagctttaaaatccctcagtgtaattgttcttcattcataacgtggcagtcaaggagcttgaagactgagtccgtctcatacccacagtatcggcatctctgtcaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacatctcggttagcgtatcccccttcctcggcttttacaaatgatgcgctcttaatagtgtgtcgttatccttttggcattgacgggggagggaaattgattgagcgcatccatattttggcggactgctgaggacaatggtggtttttccgggtggcgtgggctacaaatgatacgatggtttttttcttttcggagaaggcgtataaaaaggacacggagaacccatttattctaataacagttgagcttctttaattatttgttaatataatattctattattatatattttcttcccaataaaacaaaataaaacaaaacacagcaaaacacaaaaataatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaactctgatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattggccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaactctggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataataacccgcttgatcacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccgagcttggataggtcactctctattttcgtttctcccttccctgataggcttgaagactgagtccgtctcagactcagtatcggcatctctgtcactccaaatagtactgtagcatgtagtgatattgtttattatatcttctactaatatattaattatgtcctctactaatatattaattatgtcactacacctaaaacattttaagaattaatcccgctgagaaataaacaaaaagtatccctcaatactactcaattaggtaaattagcacccgttgcagccttaccattgccatggccagatgcacgataatgtatatagaaggacaaacttgcattagtaccaccaaggcgataagaacaccagccactgtagacgatctacacttacaaggggcaattgctaataagtaatggcaaataagcaataagcggcgaacaacgagatactattcatggagaaccttcggcaatacacgagagattatagcaatacacgagaaaccataagatactaagataaatcatggcaaatcatattaactattgacctctgacaaacagttatggccctttaaagaaggtaaaacgtgggaagccttgggacaggaaaaaaaaaaaaaaaccttctctctcaatgagccaacttttcattacatcatcatcgtccacaatttaattggacaataggaaaatgcaaaacaaataaagctgagtaaagagcggcaaaaatatgcaaaagagacaaagatttgccaaagaggcaaagatctgcagaaatgggaaaaaaaacttcataaattgcaaaacgcgcttctatttttagtacattcgacagcggccgtgctgtttatcttttgccgcttacggaaggcgcgcgccgccggtggctgttttctggtaaagtgactgttccacggggggaagctataaaaagcgtgaaatccctcccacattttctaatcccagtggtaaacccaacttcttttctatattttttttattttttttctttctcacttatcaacttttatcgttcatagtctctcgcttacaaactaaaatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaactctgatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattggccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaactctggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataataacttctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaagggcttgaagactgagtccgtctcagcgagctcttctagaataacttcgtataatgtatgctatacgaagttatcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtattccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactcaacgc

SEQ ID NO: 14 - DNA, 8747 bp, Artificial Sequence
  syntehtic
ggccgcatcgttgaaggagcttgccaagaaacataattttatgatttttgaagatagaaaatttgctgatattggtaacactgttaaaaatcaatataaatctggtgtattccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgataacttcgtataatgtatgctatacgaagttatgtcacagtatcggcatctctgtcacaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacaattacaaaaaaatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaactctgatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattggccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaactctggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataataacgcggcgaatctctggctcatgggggatatcctctttgtttggcttttttttcccattctctgttttgattatctaatgactcattgggaggattttctcacttcaagcttttttttcttgcactctttcataactccagctctctctaactgaggctacaatgccttttaacgaacttatgagatgtttctaaattatataggtatatgccaatatataattacacataaaaataaatataaataaaatataaaaataaaaataaacatcgaaaaagaagatgtgaaattgcgaagattagaaagcacaaacgagcggtctatatcggcgagcttgaagactgagtccgtctcatacccacagtatcggcatctctgtcaagcttgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatatcgacaaaggaaaagggggacggatctccgaggcctgggacccgtgggccgccgtcggacgtggttattattctaaattaccttgacataaatatttaatagacaaataatcatccaaaccatatttagaaccttctctaccgtaaccagattccttaacaccaccgaatggagcagcttctgaagcgactgcaccttcatttataccaacaataccagcttccaaagccctagcgaccctatcaattcttttgacaccttcagaataaaaataagctgctaaaccgaatggagtgtcgttagcagcgttaactgcttcagcctcgtcctcgaaacgaaacaatgggactattggaccgaaagtttcctctgagttcaactgcattgctgctgttgcgtcagccaaaacagttggagcgtagtagtgtggacccaactctgccaacctcttaccaccagtcaagactctagcaccatgtgcaacagcgtcatcgacgtgcctagcaatcttgtccaatgccctagaattaatcattggaccaatttgagcagctggatcagttgctggagcaaccttcaaagcaccaaccctttcagccaacaaagcaccaaacctctcgtagacaccagcttggacgtagacacgatttggacagacacaagtctgaccagagttacgaaacttagcagccatcaaaccagcgactgcagcctccaagtcagcatcgtcaaaaacaataaatggagcattaccacccaactccaaagataacttctttaaagtttcagcagattccctagccaagtacttaccgactggagttgatccagtaaaagtaatttttctaactctagcgtcatgcaaccactcagcgactgctgcgacagcatgatcacgggatgcagtaatcaagttcaaaacaccgggtggaacaccagcttcgtgagccaacaagaccaaagctgaagcagtcaatggagtgtcctcagctggcttagcgacaacagtacaaccagctgccaaagcgggtgctatctttcttgcaatcattgccaatggccaattccatggtgtaatagcagcaacgactccgactggctctttgacagcagtcatcctcttacccctctgttgctgaggaatcaaatcaccgtaaattcttgttgcttcctctgcgaaccatgcgacgtaagaagcaccataagcgacctcacccttaccctcagccaaaggtttaccttgctccaaagatattaaagcacccaatgcatcagagttagccattatcaaagcgtgccaacgatgcaagactgcagctctctcttttggcaatctagccctccaagctggcaaagctcttgcagctgcatcagtagcagctcttgcatcagctggagaagagtcagcgacctcagcaattatctctccagaagctgggttagtgacagcgaacctcaagttgtcttgtgctggagtccaagcaccatcaatgaagttatcagaacgaatcaattcacccaattcaaaagttctagtcatttttttctaatagggggagggggggttcgtttggatttaattgatgttaaaagaaactaaaataaaaaaatcaaagacaaaataatataattggaaaaataacaaatacacatcaaccaatatacatggcggcaaatgggtgtccttttataagagttggtgtacaatttgttatcttgactatgatgccacccaccctcacattgaggcccaccaaaggtgaaaaccatcaccctaccctcgcgccgacgcaccccgtgtatggaataattgggaattgaccatttactgcaacaatatcactagcgccattagtaacaatagaattgggactattttctatccgacaatttccagaacttcctcattgtagaccactagcccccgttgggtaataacgaaaatgtaaaacaataccaccgaacggactccggcgtttcccgcactcatgcccccccgcagaagtctcgcccgggggtgggaacattattctcaacgtagaggctgcaaacagggggggcttgaagactgagtccgtctcagactcagtatcggcatctctgtcacatagaaggtatttgaaacatcatgaaaactgtttcaccctctgtgaagcataaacactagaaagccaatggagagctctacaagcctcatatgggttcaatgggtctgcaatgaccgcatacggacttggacaattaccttctattgaatttctgagaagagatacatctgaccagcaatgtaagcagacaatcccaattctgtaaacaacctctttgtccataattccccatcagaagagtgaaaaatgccctcaaaacgcatgcgccactcccacctctcagctgcactgcgccacctctgagggtcctttcaggggtcgactaccccggacacctcgcagaggagcgagatcacgtacttttaaaatggcagagatgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttccttgcgttttcttttcaactttttttactttttccttcaacagacaaatctaacttatatatcacaaatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaactctgatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattggccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaactctggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataataacgtatagccatatagtttaattcctttatactttttataactatttcttacactaattattattatcaattatttattgtagaacttgactcttgcgtcgatcaccatgacagggctatcttaacaaggggtaatttttgttgatggagtcaagtagcattccgacgggaagtgtcgatgcctctgaacgaaatcttccgattagctctgcaaagaagtggaaattgtcagcgcgcttgaagactgagtccgtctcagcgagctcttctagaatttgatataaacgcttctataataataaataacatcaacaagtgactaaccccaagtatccaattttagatctaatgcctgaaagtgccttgcacgatttaggattcggcaccagaaagtttgtgccgtgctcgcttattggacttgtatagtgataaggcaaaaaaaaaaaacttgaaagtacttgcgtaactcagaggttgccttttcttatcttggagtcaaaagagattcttttaattaacaaatgaagaacacagtaccgctattaattgcttctgcagtgtgtgtcactgccaacattatcccagaaggtttccatgttagggaaaggttgcaaaatgttttaggcaataaccattatcatccaattcttgatgagtatcttcaaagctccacagagccatctgactttggatctttaacaccagaagtttcggaggcatgggaatatttggagaagrcattcgataggtcggttatagaagatgccattaagcaacgc

SEQ ID NO: 15 - DNA, 9580 bp, Artificial Sequence
  synthetic
ggccgcatcgaccagcgccagcaccaccagcgccatctttgattccttcaatcacaggtggcccagcagatatcaatgctttacttggggaaatccatcaaggtgcaaggttgaagaaggtcgacgacagcgagaaacacattgctgacggtgccgttgttggcagagtattgtaattgcaacatgttcctgtttagtctagcatatatatacttacttataaagctaaaatattgcgttttgttctagttttaactgctgtctctttcttgccgagccccctttattttcttcctttagagaggcaataatatttcgataagcgcaatcccgaattttttctgcggcgagagagatgcgggactgggcttccgaagaacgtaaccaaactgattttatccaatataaaagagagggtgccacagctgtttgcaggagaaggtactcttatttacacggtagtctcctctcacatttcagatataatacaagcaatattcaacagcagacgtcacagtatcggcatctctgtcatccttgactgccacgttatgaatgaagaacaattacactgagggattttaaagctgccctgtagagtgatgtccttttgttaaagtctgttacactacaacaatggttttgaaaaaaaaacccattgagaaattaaccaaagtccgggattatcggaagaacgcaacttaataaacacatcgttacaaaatatttttttttcattaaattcatttataaacgtagggactgtaaaaattgtactagatatttagtaaaagcattagttagatttagttattattctaaattaccttgacataaatatttaatagacaaataatcatccaaaccatatttagaaccttctctaccgtaaccagattccttaacaccaccgaatggagcagcttctgaagcgactgcaccttcatttataccaacaataccagcttccaaagccctagcgaccctatcaattcttttgacaccttcagaataaaaataagctgctaaaccgaatggagtgtcgttagcagcgttaactgcttcagcctcgtcctcgaaacgaaacaatgggactattggaccgaaagtttcctctgagttcaactgcattgctgctgttgcgtcagccaaaacagttggagcgtagtagtgtggacccaactctgccaacctcttaccaccagtcaagactctagcaccatgtgcaacagcgtcatcgacgtgcctagcaatcttgtccaatgccctagaattaatcattggaccaatttgagcagctggatcagttgctggagcaaccttcaaagcaccaaccctttcagccaacaaagcaccaaacctctcgtagacaccagcttggacgtagacacgatttggacagacacaagtctgaccagagttacgaaacttagcagccatcaaaccagcgactgcagcctccaagtcagcatcgtcaaaaacaataaatggagcattaccacccaactccaaagataacttctttaaagtttcagcagattccctagccaagtacttaccgactggagttgatccagtaaaagtaatttttctaactctagcgtcatgcaaccactcagcgactgctgcgacagcatgatcacgggatgcagtaatcaagttcaaaacaccgggtggaacaccagcttcgtgagccaacaagaccaaagctgaagcagtcaatggagtgtcctcagctggcttagcgacaacagtacaaccagctgccaaagcgggtgctatctttcttgcaatcattgccaatggccaattccatggtgtaatagcagcaacgactccgactggctctttgacagcagtcatcctcttacccctctgttgctgaggaatcaaatcaccgtaaattcttgttgcttcctctgcgaaccatgcgacgtaagaagcaccataagcgacctcacccttaccctcagccaaaggtttaccttgctccaaagatattaaagcacccaatgcatcagagttagccattatcaaagcgtgccaacgatgcaagactgcagctctctcttttggcaatctagccctccaagctggcaaagctcttgcagctgcatcagtagcagctcttgcatcagctggagaagagtcagcgacctcagcaattatctctccagaagctgggttagtgacagcgaacctcaagttgtcttgtgctggagtccaagcaccatcaatgaagttatcagaacgaatcaattcacccaattcaaaagttctagtcatttgtttggttggagggggttatatgtgtaaatatagagtttgtgtttggttgtgtattgatggacactggggaaagttatctctctcttctaaagttccaacttcttcctctctctgggggtggacatctcctgtctttatataccacccccccccctcatagtctggaactcttccccgtccgagagcgacgtgattttccctatatttctccttccaaattgccccattgaggcaaccgcgtgatacgccgggtcttttcttcccgggagtggagacagttcccaagtggtaaagtccacttccatcatgatccacctccatgacactcttttaaacacatgtgctgcacatgtgttggtgtggttacacgatatacaaccatgttgggcaaccacaggtgcgcagcatcaacaacggggcgagtcaggaggaacggtaaccttggcggtttacgaatgaggtgttccccgttggagaggtgtctatggacgaccatcccagtacaatggacgggttggttggtgtggttacggtgtcaacgacagagacatgggccaacagagacattatggagagatgttgggagtggatcctccctggaaagggtgtctttggcgaagtgttgttgatttgtatgtctgggtcggttgtttgttgtgctagttgctgcataatggtaccaacaccagcggtttttttgtcctcctctgcaacaggggaactcggccacttgtttattgggggggggggggggatgccacgtggtaatgaaacattatcaagagatctctctttgtaggggtacctgtgatgtgtgtgtgtgtgcatgtgcatagatgcatattgagacagttgtgttcattgactgtctgcatttctccagaaccaataaaaaataaaaatagtagcaatacctacactttctggaccttgtagtaacgtagacgccgtaaggttgcatttcacgccagcttgagagggggaaagagcttgaagactgagtccgtctcatacccacagtatcggcatctctgtcactatcagggaagggagaaacgaaaatagagagtgacctatccaagctcgggggtctaagttttaatggcccagggaatcattacttttttttctcaatccttgatggataaaagtattacatacgtacaggattgtgtattagtgtatttcgttatatgattaaacaaagtttatagattgtaaagtagacgtaaagtttagtaattcattttaatgttcattttacattcagatgtgatcaagcgggttattattctaaattaccttgacataaatatttaatagacaaataatcatccaaaccatatttagaaccttctctaccgtaaccagattccttaacaccaccgaatggagcagcttctgaagcgactgcaccttcatttataccaacaataccagcttccaaagccctagcgaccctatcaattcttttgacaccttcagaataaaaataagctgctaaaccgaatggagtgtcgttagcagcgttaactgcttcagcctcgtcctcgaaacgaaacaatgggactattggaccgaaagtttcctctgagttcaactgcattgctgctgttgcgtcagccaaaacagttggagcgtagtagtgtggacccaactctgccaacctcttaccaccagtcaagactctagcaccatgtgcaacagcgtcatcgacgtgcctagcaatcttgtccaatgccctagaattaatcattggaccaatttgagcagctggatcagttgctggagcaaccttcaaagcaccaaccctttcagccaacaaagcaccaaacctctcgtagacaccagcttggacgtagacacgatttggacagacacaagtctgaccagagttacgaaacttagcagccatcaaaccagcgactgcagcctccaagtcagcatcgtcaaaaacaataaatggagcattaccacccaactccaaagataacttctttaaagtttcagcagattccctagccaagtacttaccgactggagttgatccagtaaaagtaatttttctaactctagcgtcatgcaaccactcagcgactgctgcgacagcatgatcacgggatgcagtaatcaagttcaaaacaccgggtggaacaccagcttcgtgagccaacaagaccaaagctgaagcagtcaatggagtgtcctcagctggcttagcgacaacagtacaaccagctgccaaagcgggtgctatctttcttgcaatcattgccaatggccaattccatggtgtaatagcagcaacgactccgactggctctttgacagcagtcatcctcttacccctctgttgctgaggaatcaaatcaccgtaaattcttgttgcttcctctgcgaaccatgcgacgtaagaagcaccataagcgacctcacccttaccctcagccaaaggtttaccttgctccaaagatattaaagcacccaatgcatcagagttagccattatcaaagcgtgccaacgatgcaagactgcagctctctcttttggcaatctagccctccaagctggcaaagctcttgcagctgcatcagtagcagctcttgcatcagctggagaagagtcagcgacctcagcaattatctctccagaagctgggttagtgacagcgaacctcaagttgtcttgtgctggagtccaagcaccatcaatgaagttatcagaacgaatcaattcacccaattcaaaagttctagtcattatttttgtgttttgctgtgttttgttttattttgttttattgggaagaaaatatataataatagaatattatattaacaaataattaaagaagctcaactgttattagaataaatgggttctccgtgtcctttttatacgccttctccgaaaagaaaaaaaccatcgtatcatttgtagcccacgccacccggaaaaaccaccattgtcctcagcagtccgccaaaatatggatgcgctcaatcaatttccctcccccgtcaatgccaaaaggataacgacacactattaagagcgcatcatttgtaaaagccgaggaagggggatacgctaaccgagatgtctcgcctcactctcggagctgagccgccctccttaagaaattcatgggaagaacacccttcgcggcttctgaacggctcgccctcgtccattggtcacctcacagtggcaactaataaggacattatagcaatagaaattaaaatggtgcactgaaatacaataggatcgaataggataggatacaataagatacggaatattagactatactgtgatacggtacgctacgatacgctacgatacgatacgatagaggataccacggatataacgtagtgttatttttcattattggggtgcttgaagactgagtccgtctcagactcagtatcggcatctctgtcactccaaatagtactgtagcatgtagtgatattgtttattatatcttctactaatatattaattatgtcctctactaatatattaattatgtcactacacctaaaacattttaagaattaatcccgctgagaaataaacaaaaagtatccctcaatactactcaattaggtaaattagcacccgttgcagccttaccattgccatggccagatgcacgataatgtatatagaaggacaaacttgcattagtaccaccaaggcgataagaacaccagccactgtagacgatctacacttacaaggggcaattgctaataagtaatggcaaataagcaataagcggcgaacaacgagatactattcatggagaaccttcggcaatacacgagagattatagcaatacacgagaaaccataagatactaagataaatcatggcaaatcatattaactattgacctctgacaaacagttatggccctttaaagaaggtaaaacgtgggaagccttgggacaggaaaaaaaaaaaaaaaccttctctctcaatgagccaacttttcattacatcatcatcgtccacaatttaattggacaataggaaaatgcaaaacaaataaagctgagtaaagagcggcaaaaatatgcaaaagagacaaagatttgccaaagaggcaaagatctgcagaaatgggaaaaaaaacttcataaattgcaaaacgcgcttctatttttagtacattcgacagcggccgtgctgtttatcttttgccgcttacggaaggcgcgcgccgccggtggctgttttctggtaaagtgactgttccacggggggaagctataaaaagcgtgaaatccctcccacattttctaatcccagtggtaaacccaacttcttttctatattttttttattttttttctttctcacttatcaacttttatcgttcatagtctctcgcttacaaactaaaatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaactctgatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattggccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaactctggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataataacttctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaagggcttgaagactgagtccgtctcagcgagctcttctagaataacttcgtataatgtatgctatacgaagttatcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtattccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactcaacgc

SEQ ID NO: 16 - DNA, 8747 bp, Artificial Sequence
  synthetic
ggccgcatcgttgaaggagcttgccaagaaacataattttatgatttttgaagatagaaaatttgctgatattggtaacactgttaaaaatcaatataaatctggtgtattccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgataacttcgtataatgtatgctatacgaagttatgtcacagtatcggcatctctgtcacaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacaattacaaaaaaatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaactctgatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattggccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaactctggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataataacgcggcgaatctctggctcatgggggatatcctctttgtttggcttttttttcccattctctgttttgattatctaatgactcattgggaggattttctcacttcaagcttttttttcttgcactctttcataactccagctctctctaactgaggctacaatgccttttaacgaacttatgagatgtttctaaattatataggtatatgccaatatataattacacataaaaataaatataaataaaatataaaaataaaaataaacatcgaaaaagaagatgtgaaattgcgaagattagaaagcacaaacgagcggtctatatcggcgagcttgaagactgagtccgtctcatacccacagtatcggcatctctgtcacatagaaggtatttgaaacatcatgaaaactgtttcaccctctgtgaagcataaacactagaaagccaatggagagctctacaagcctcatatgggttcaatgggtctgcaatgaccgcatacggacttggacaattaccttctattgaatttctgagaagagatacatctgaccagcaatgtaagcagacaatcccaattctgtaaacaacctctttgtccataattccccatcagaagagtgaaaaatgccctcaaaacgcatgcgccactcccacctctcagctgcactgcgccacctctgagggtcctttcaggggtcgactaccccggacacctcgcagaggagcgagatcacgtacttttaaaatggcagagatgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttccttgcgttttcttttcaactttttttactttttccttcaacagacaaatctaacttatatatcacaaatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaactctgatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattggccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaactctggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataataacgtatagccatatagtttaattcctttatactttttataactatttcttacactaattattattatcaattatttattgtagaacttgactcttgcgtcgatcaccatgacagggctatcttaacaaggggtaatttttgttgatggagtcaagtagcattccgacgggaagtgtcgatgcctctgaacgaaatcttccgattagctctgcaaagaagtggaaattgtcagcgcgcttgaagactgagtccgtctcagactcagtatcggcatctctgtcaagcttgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatatcgacaaaggaaaagggggacggatctccgaggcctgggacccgtgggccgccgtcggacgtggttattattctaaattaccttgacataaatatttaatagacaaataatcatccaaaccatatttagaaccttctctaccgtaaccagattccttaacaccaccgaatggagcagcttctgaagcgactgcaccttcatttataccaacaataccagcttccaaagccctagcgaccctatcaattcttttgacaccttcagaataaaaataagctgctaaaccgaatggagtgtcgttagcagcgttaactgcttcagcctcgtcctcgaaacgaaacaatgggactattggaccgaaagtttcctctgagttcaactgcattgctgctgttgcgtcagccaaaacagttggagcgtagtagtgtggacccaactctgccaacctcttaccaccagtcaagactctagcaccatgtgcaacagcgtcatcgacgtgcctagcaatcttgtccaatgccctagaattaatcattggaccaatttgagcagctggatcagttgctggagcaaccttcaaagcaccaaccctttcagccaacaaagcaccaaacctctcgtagacaccagcttggacgtagacacgatttggacagacacaagtctgaccagagttacgaaacttagcagccatcaaaccagcgactgcagcctccaagtcagcatcgtcaaaaacaataaatggagcattaccacccaactccaaagataacttctttaaagtttcagcagattccctagccaagtacttaccgactggagttgatccagtaaaagtaatttttctaactctagcgtcatgcaaccactcagcgactgctgcgacagcatgatcacgggatgcagtaatcaagttcaaaacaccgggtggaacaccagcttcgtgagccaacaagaccaaagctgaagcagtcaatggagtgtcctcagctggcttagcgacaacagtacaaccagctgccaaagcgggtgctatctttcttgcaatcattgccaatggccaattccatggtgtaatagcagcaacgactccgactggctctttgacagcagtcatcctcttacccctctgttgctgaggaatcaaatcaccgtaaattcttgttgcttcctctgcgaaccatgcgacgtaagaagcaccataagcgacctcacccttaccctcagccaaaggtttaccttgctccaaagatattaaagcacccaatgcatcagagttagccattatcaaagcgtgccaacgatgcaagactgcagctctctcttttggcaatctagccctccaagctggcaaagctcttgcagctgcatcagtagcagctcttgcatcagctggagaagagtcagcgacctcagcaattatctctccagaagctgggttagtgacagcgaacctcaagttgtcttgtgctggagtccaagcaccatcaatgaagttatcagaacgaatcaattcacccaattcaaaagttctagtcatttttttctaatagggggagggggggttcgtttggatttaattgatgttaaaagaaactaaaataaaaaaatcaaagacaaaataatataattggaaaaataacaaatacacatcaaccaatatacatggcggcaaatgggtgtccttttataagagttggtgtacaatttgttatcttgactatgatgccacccaccctcacattgaggcccaccaaaggtgaaaaccatcaccctaccctcgcgccgacgcaccccgtgtatggaataattgggaattgaccatttactgcaacaatatcactagcgccattagtaacaatagaattgggactattttctatccgacaatttccagaacttcctcattgtagaccactagcccccgttgggtaataacgaaaatgtaaaacaataccaccgaacggactccggcgtttcccgcactcatgcccccccgcagaagtctcgcccgggggtgggaacattattctcaacgtagaggctgcaaacagggggggcttgaagactgagtccgtctcagcgagctcttctagaatttgatataaacgcttctataataataaataacatcaacaagtgactaaccccaagtatccaattttagatctaatgcctgaaagtgccttgcacgatttaggattcggcaccagaaagtttgtgccgtgctcgcttattggacttgtatagtgataaggcaaaaaaaaaaaacttgaaagtacttgcgtaactcagaggttgccttttcttatcttggagtcaaaagagattcttttaattaacaaatgaagaacacagtaccgctattaattgcttctgcagtgtgtgtcactgccaacattatcccagaaggtttccatgttagggaaaggttgcaaaatgttttaggcaataaccattatcatccaattcttgatgagtatcttcaaagctccacagagccatctgactttggatctttaacaccagaagtttcggaggcatgggaatatttggagaagrcattcgataggtcggttatagaagatgccattaagcaacgc

SEQ ID NO: 17 - DNA, 1512 bp, Artificial Sequence
  synthetic
ggccgcacaacatggttaaactattgctatggtgatggtattacagatagtaaaagaaggaagggggggtggcaatctcaccctaacagttactaagaacgtctacttcatctactgrcaatatacattggccacatgccgagaaattacgtcgacgccaaagaagggcccagccgaaaaaagaaatggaaaacttggccgaaaagggaaacaaacaaaaaggtgatgtaaaattagcggaaaggggaattggcaaattgagggagaaaaaaaaaaggcacaaaaggaggcggaaagtcagtacgttttgaaggcgtcattggttttcccttttgcagagtgtttcatttcttttgtttaatgacgtagtggcgtttcttttcctgcactttagaaatctatcttttccttatcaagtaacaagcggttggcaaaggtgtatataaatcaaggaattcccactttgaaccctttgaattttgatatcgtttattttaaatttattttgtcatgagaccggtctcagcttgaagactgagtccgtctcagcgagctcttctagaataacttcgtataatgtatgctatacgaagttatcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtattccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactgc

SEQ ID NO: 18 - DNA, 1439 bp, Artificial Sequence
  synthetic
ggccgcattgaaggagcttgccaagaaacataattttatgatttttgaagatagaaaatttgctgatattggtaacactgttaaaaatcaatataaatctggtgtattccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgataacttcgtataatgtatgctatacgaagttatgtcatgagaccggtctcagcttgaagactgagtccgtctcagcgagctcttctagaataacctcatggagaactttggcattgtactctccattgacgagtccgcraacccattcttgttaaacctaaccttgcattatcacattccctttgaccccctttagctgcatttccacttgtctacattaagattcattacacattctttttcgtatttctcttacctccctcccccctccatggatcttatatataaatcttttctataacaataatatctactagagttaaacaacaattccacttgccatggctgtctcagcaaatctgcttctacctactgcacgggtttgcatgtcattgtttctagcagggaatcgtccatgtacgttgtcctccatgatggtcttaccgctgccactttctttagtatcttaaatagagcagatcttacgtccacagtgcatccgtgcaccccgaaaatcgtatggttttccttgccacctctcacaattttgaatatgctcaacgcgaaagagaggggaagaggaatcgcagc

SEQ ID NO: 19 - DNA, 1921 bp, Artificial Sequence
  synthetic
ggccgcacaacatggttaaactattgctatggtgatggtattacagatagtaaaagaaggaagggggggtggcaatctcaccctaacagttactaagaacgtctacttcatctactgrcaatatacattggccacatgccgagaaattacgtcgacgccaaagaagggcccagccgaaaaaagaaatggaaaacttggccgaaaagggaaacaaacaaaaaggtgatgtaaaattagcggaaaggggaattggcaaattgagggagaaaaaaaaaaggcacaaaaggaggcggaaagtcagtacgttttgaaggcgtcattggttttcccttttgcagagtgtttcatttcttttgtttaatgacgtagtggcgtttcttttcctgcactttagaaatctatcttttccttatcaagtaacaagcggttggcaaaggtgtatataaatcaaggaattcccactttgaaccctttgaattttgatatcgtttattttaaatttattttgtcatgagaccggtctcagcttgaagactgagtccgtctcagcgagctcttctagaataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgtaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaatctagatgaaaaagcctgaactcaccgcgacgtctgtcgagaagtttctgatcgaaaagttcgacagcgtatccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatctgc

SEQ ID NO: 20 - DNA, 1565 bp, Artificial Sequence
  synthetic
ggccgcagatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagaaattcggatccggtagatacattgatgctatcaatccagagaactggaaagattgtgtagccttgaaaaacggtgaaacttacgggtccaagattgtctacagattttcctgatttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccataacttcgtataatgtatgctatacgaagttatgtcatgagaccggtctcagcttgaagactgagtccgtctcagcgagctcttctagaataacctcatggagaactttggcattgtactctccattgacgagtccgcraacccattcttgttaaacctaaccttgcattatcacattccctttgaccccctttagctgcatttccacttgtctacattaagattcattacacattctttttcgtatttctcttacctccctcccccctccatggatcttatatataaatcttttctataacaataatatctactagagttaaacaacaattccacttgccatggctgtctcagcaaatctgcttctacctactgcacgggtttgcatgtcattgtttctagcagggaatcgtccatgtacgttgtcctccatgatggtcttaccgctgccactttctttagtatcttaaatagagcagatcttacgtccacagtgcatccgtgcaccccgaaaatcgtatggttttccttgccacctctcacaattttgaatatgctcaacgcgaaagagaggggaagaggaatcgcagc

SEQ ID NO: 21 - DNA, 9629 bp, Artificial Sequence
  synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggtcgaggagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaaggatccatgtctaatttacttactgttcaccaaaacttgcctgcattaccagttgacgcaacctccgatgaagtcagaaagaaccttatggatatgtttagagatagacaagctttctccgaacatacttggaaaatgttattatccgtttgtagatcctgggccgcttggtgtaaacttaacaatagaaaatggtttcctgctgaaccagaagacgtcagagattacttactttacttacaagctagaggtttggctgttaaaactatccaacaacacttaggtcaattgaatatgttacacagaagatccggtttaccaagaccatccgattccaacgcagtttcccttgttatgagaagaattagaaaagaaaatgttgacgctggtgaaagagctaaacaagcattagcatttgaaagaaccgatttcgatcaagttagatccttaatggaaaattccgatagatgtcaagatattagaaacttagctttcttaggtattgcttacaacacattattaagaatcgctgaaattgctagaattagagttaaagatatttcaagaaccgatggcggtagaatgttaatccacattggcagaacaaaaaccttagtctccacagcaggcgtcgaaaaagcattatcattaggtgttactaaattagttgaacgttggatttccgtttccggtgttgcagatgacccaaacaactacttattctgtcgtgttagaaaaaatggtgttgccgctccttccgctacctcacaattatccacaagagcattagaaggcatttttgaagctacccacagacttatttatggtgcaaaagacgattccggtcaaagatatttagcttggtctggtcattccgctagagttggtgccgcaagagacatggcaagagctggtgtttctattcctgaaattatgcaagccggtggttggactaatgttaacattgttatgaactatatcagaaacttagattccgaaacaggtgctatggttagattacttgaagacggtgattaagttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcggcagatctggatcgatcccccgggctgcatgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgaaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacctgcaggaaatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagcctgcaggcacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgaattcccgggccttaccgtcgacgaatttcagcattttcatttcaaggcgatattatgtttcactaaactcaggacaggaatatactaagaataactacaacatacacacaacataagccaagatggatcaacttaactaccaagaacaacaacaatttcaaaagatcgttgaacaaaagcaaatggctgatttcatgaggctatgaattcgcccttgatctgggtgtatactgcacaacctcattgttcgggaatttgattctcatctcacatacaggcctgtagtattgcgccctctccttctccttctccttctccttctccaagagagacttctctctcatcgccctcgtcatcaatggctgctcgctgtattgtcgttggagcatctcccgatacttctgcaactgtgataaactcatctcaggtgacccatccgattctgtatcggtgtctccatctggggctacatctcgggccagtctagatttaaactttgcagaaccttcactttgggggatatacactagtgtctctcccgtgactacatcaccgacaccctcaactgtaccattattattgtcattgttttcctctaagttctcgctttggtcttcatccatctctccttcgggtgctgtatcactcttgatgatttctctaaccctaatacggagactgtgattgcctgaaataatacccacatctttcaacttctgatgaagtgaatctccagagatgaccttcatcagcacttgcacatcaaccacatcaccctccttttgagcatccctcatgattccatagactacatcccgtagcgtctccttgttcttgtacttcttaacaacagtctcgccacagacatggcccctgataatcacctcctgtctctcctcatggccatcctggtcgccattgtcttcgtcgctcggctcaattgccaatgtagcaccctgtggaagattgcttagtctgtatggaacagactcatcaactcttttgccattatgcattaacttgtactttcgcccttggctaagttgaaaatgtttacatccttcttcaagtacattggacatgattgtccctgcattgacatttgtccggtaagtcctaaacccactcgctagattcactgtaggcatattcaatcacgttccgtttgaaaaaaaggaaaccaatttattatctccagaaatagttggcgtcttgcatcttgtttggtcttgatctttcgtgttttttttttttctgtctttttttttctcctctctccaactttttgatttttagtgtaccaaatcgcactgcttatccacattcatcataaagagggggggagaagaggggcagaaaataaaaggccatgtcacgtgcctgtgcatttatttgtgtgtgtgtcacgtgctcaaaatgtcttttttttacgtttttaacattttccctttctgtagttgaatccatttgcatgagtcgtacatgatgtttgctgtatttacgttaagacactaattcaaatgacaaacagctattattcttagccattaatgcatttttgcaaatctttaactggatttaactatggctaggtgaatttgttctggacatcattgccttgacttgttttagtgccgatgtccttatcacttacactcgtaacacaacacaacagcagctaatgttgttgtgtatcgcttgacccttaataactgattcttttttgatgaatgttaagaagaaacaaacaagaaaataaaatcaaaacaggcttcttttgacctctttcaagagaaggttttcttggttgtttcatataccaagatctgaatatcttctattattatacaaaccactgattatacaaatctattcatcgacagtatgagctacgaaaacacactgataaagagagtcatttcttccccttctttttctttttctttttcttcttcttcttagtatccccatcttcattaactccaccaagtagatcctctacaccccccatggccgttaaaaaatgttcacgaaagaaatccatatcattattcttaccatccattaaactgtttagatagatggtgatcatctcccttgcattgtctatatcttcaacgtcgagtaaatgcgacgcaatggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 22 - protein, 508 aa, Danaus plexippus
MRVDSKIIVKKETQDENLYQSLAERSKHEEFLRKAVDLLVERVVFGRSTRSSKVVEWAAPDEIKKAIDLKPRLGPASHDELLAFMANVARYSVNTGHPYFVNQLFSSVDPYGLVGQWLTDALNPSVYTFEVAPVFTLMEEEVLREMRKIVGWPEGEGDGIFCPGGSIANGYAISCARHHFYPEVKYKGVHAVPKLVLFTSELAHYSTKKMAAFMGIGSDNCVNIKTDDVGKMNIVDLEMKIKIAIDNKCTPFMVTATSGTTVFGAFDPLVAISDLCKKYNLWLHVDAAWGGGALMSKKHRHLLNGIELADSVTWNPHKLLAAPQQCSTFLTRHKKVLSEGHSSNAKYLFQKDKFYDTSYDTGDKHIQCGRRADVLKFWFMWKAKGTEGFEKHVDKLFDNAKYFLDHIKQREGFQLVIAEPQCTNIMFWYIPKCLRGCENDADYYERLHKVAPKIKERMIKEGSMMVTYQPQGDLVNFFRIVFQNSALDHKDMVYFANEFERLGSDMIV

SEQ ID NO: 23 - protein, 1180 aa, Pichia kudriavzevii
MSTVEDHSSLHKLRKESEILSNANKILVANRGEIPIRIFRSAHELSMHTVAIYSHEDRLSMHRLKADEAYAIGKTGQYSPVQAYLQIDEIIKIAKEHDVSMIHPGYGFLSENSEFAKKVEESGMIWVGPPAEVIDSVGDKVSARNLAIKCDVPVVPGTDGPIEDIEQAKQFVEQYGYPVIIKAAFGGGGRGMRVVREGDDIVDAFQRASSEAKSAFGNGTCFIERFLDKPKHIEVQLLADNYGNTIHLFERDCSVQRRHQKVVEIAPAKTLPVEVRNAILKDAVTLAKTANYRNAGTAEFLVDSQNRHYFIEINPRIQVEHTITEEITGVDIVAAQIQIAAGASLEQLGLLQNKITTRGFAIQCRITTEDPAKNFAPDTGKIEVYRSAGGNGVRLDGGNGFAGAVISPHYDSMLVKCSTSGSNYEIARRKMIRALVEFRIRGVKTNIPFLLALLTHPVFISGDCWTTFIDDTPSLFEMVSSKNRAQKLLAYIGDLCVNGSSIKGQIGFPKLNKEAEIPDLLDPNDEVIDVSKPSTNGLRPYLLKYGPDAFSKKVREFDGCMIMDTTWRDAHQSLLATRVRTIDLLRIAPTTSHALQNAFALECWGGATFDVAMRFLYEDPWERLRQLRKAVPNIPFQMLLRGANGVAYSSLPDNAIDHFVKQAKDNGVDIFRVFDALNDLEQLKVGVDAVKKAGGVVEATVCYSGDMLIPGKKYNLDYYLETVGKIVEMGTHILGIKDMAGTLKPKAAKLLIGSIRSKYPDLVIHVHTHDSAGTGISTYVACALAGADIVDCAINSMSGLTSQPSMSAFIAALDGDIETGVPEHFARQLDAYWAEMRLLYSCFEADLKGPDPEVYKHEIPGGQLTNLIFQAQQVGLGEQWEETKKKYEDANMLLGDIVKVTPTSKVVGDLAQFMVSNKLEKEDVEKLANELDFPDSVLDFFEGLMGTPYGGFPEPLRTNVISGKRRKLKGRPGLELEPFNLEEIRENLVSRFGPGITECDVASYNMYPKVYEQYRKVVEKYGDLSVLPTKAFLAPPTIGEEVHVEIEQGKTLIIKLLAISDLSKSHGTREVYFELNGEMRKVTIEDKTAAIETVTRAKADGHNPNEVGAPMAGVVVEVRVKHGTEVKKGDPLAVLSAMKMEMVISAPVSGRVGEVFVNEGDSVDMGDLLVKIAKDEAPAA

SEQ ID NO: 24 - protein, 419 aa, Pichia kudriavzevii
MSRGFFTENITQLPPDPLFGLKARFSNDSRENKVDLGIGAYRDDNGKPWILPSVRLAENLIQNSPDYNHEYLPIGGLADFTSAAARVVFGGDSKAISQNRLVSIQSLSGTGALHVAGLFIKRQYKSLDGTSEDPLIYLSEPTWANHVQIFEVIGLKPVFYPYWHAASKTLDLKGYLKAINDAPEGSVFVLHATAHNPTGLDPTQEQWMEILAAISAKKHLPLFDCAYQGFTSGSLDRDAWAVREAVNNDKYEFPGIIVCQSFAKNVGMYGERIGAVHIVLPESDASLNSAIFSQLQKTIRSEISNPPGYGAKIVSKVLNTPELYKQWEQDLITMSSRITAMRKELVNELERLGTPGTWRHITEQQGMFSFTGLNPEQVAKLEKEHGVYLVRSGRASIAGLNMGNVKYVAKAIDSVVRDL

SEQ ID NO: 25 - protein, 467 aa, Pichia kudriavzevii
MSISEKYFPQEPQSPELKTAIPGPQSKAKLEELSAVYDTKAAYFVTDYYKSLGNYIVDADGNKLLDSYCQISSIALGYNNPALLKVAHSDEMAVALCNRPALACFPSTDYYEILKKGLLSVAPKGLDKVCTAHTGSDANEMAFKAAFLFQASKKRGDKPFTSEELESVMENKLPGTSDMVILSFEKGFHGRLFGSLSTTRSKAIHKLDIPAFEWPKAPFPQLKYPLDQFQAENKAEEERCLKALEEIIVNSPAKIAAAIIEPVQSEGGDNHASPEFFQGIREITKKHGVILIVDEVQTGGGASGKMWLHEHYGIVPDIMTFSKKMQNAGFFFSEAGLAGDQPFRQFNTWCGDPSKALIARTIIEEIKDKNLLTSVTETGDYLYSKLEAISAKYDKMINLRGKGRGFFIAFDAPTPELRNKFIAECKKLGLNIGGCGEQGVRLRPALVFEKKHADILASIIDQAFSKI

SEQ ID NO: 26 - protein, 490 aa, Artificial Sequence
  synthetic
MTRTFELGELIRSDNFIDGAWTPAQDNLRFAVTNPASGEIIAEVADSSPADARAATDAAARALPAWRARLPKERAAVLHRWHALIMANSDALGALISLEQGKPLAEGKGEVAYGASYVAWFAEEATRIYGDLIPQQQRGKRMTAVKEPVGVVAAITPWNWPLAMIARKIAPALAAGCTVVAKPAEDTPLTASALVLLAHEAGVPPGVLNLITASRDHAVAAVAEWLHDARVRKITFTGSTPVGKYLARESAETLKKLSLELGGNAPFIVFDDADLEAAVAGLMAAKFRNSGQTCVCPNRVYVQAGVYERFGALLAERVGALKVAPATDPAAQIGPMINSRALDKIARHVDDAVAHGARVLTGGKRLAELGPHYYAPTVLADATAAMQLNSEETFGPIVPLFRFEDEAEAVNAANDTPFGLAAYFYSEGVKRIDRVARALEAGIVGINEGAVASEAAPFGGVKESGYGREGSKYGLDDYLSIKYLCQGNLE

SEQ ID NO: 27 - protein, 490 aa, Artificial Sequence
  synthetic
MTRTFELGELIRSDNFIDGAWIPAQDNLRFAVTNPASGEIIAEVADSSPADARAATDAAARALPAWRVRLPKERAAVLHRWHALIMANSDALGALISLEQGKPLAQGKGEVAYGASYVAWFAEEATRIYGDLIPQQQRGKRMTAVKEPVGVVAAITPWNWPLAMIARKIAPALAAGCTVVAKPAEDTPLTASALVLLAHEAGVPPGVLNLITASRDHAVAAVAEWLHDARVRKITFTGSTPVGKYLARESAETLKKLSLELGGNAPFIVFDDADLEAAVAGLMAAKFRNSGQTCVCPNRVYVQADVYERFGALLAERVGALKVAPATDPAAQIGPMINSRALDKIARHVDDAVAHGARVLTGGKRLAELGPHYYAPTVLADATAAMQLNSEETFGPIVPLFRFEDEAEAVNAANNTPFGLAAYFYSEGVKRIDRVARALEAGIVGINEGAVASEAAPFGGVKESGYGREGSKYGLDDYLSIKYLCQGNLE

(19) United States
(12) Patent Application Publication
(10) Pub. No.: US 2025/0290082 A1
(43) Pub. Date: Sep. 18, 2025

(54) FERMENTATION METHOD FOR PRODUCING MALONIC ACID

(71) Applicant: CARGILL, INCORPORATED, Wayzata, MN (US)

(72) Inventors: Peter Alan JAUERT, Minneapolis, MN (US); Naga Sirisha PARIMI, Minnetonka, MN (US); Brian Jeffrey RUSH, Excelsior, MN (US)

(73) Assignee: CARGILL, INCORPORATED, Wayzata, MN (US)

(21) Appl. No.: 18/564,939

(22) PCT Filed: May 28, 2021
(86) PCT No.: PCT/US2021/034895

§ 371 Date: Nov. 28, 2023

(51) Int. Cl. C12N 15/81; C12N 9/02; C12P 7/46; C12R 1/84

(52) U.S. Cl. CPC C12N 15/815; C12N 9/0008; C12P 7/46; C12R 2001/84; C12Y 102/01015

(57) ABSTRACT

Various embodiments disclosed relate to a fermentation method for producing malonate. The fermentation method includes a) culturing a microorganism in the presence of a fermentation medium. The fermentation medium includes at least one carbon source; calcium ions ranging from 0.05 g/L to 1.6 g/L; and a base other than a calcium containing base. The fermentation method further comprises b) producing at least 40 grams/liter of malonate. Optionally, the malonate is produced at a rate of at least 0.3 g L⁻¹ h⁻¹ to 5 g L⁻¹ h⁻¹.

### CROSS-REFERENCE TO RELATED APPLICATIONS

This application is a national phase application of PCT Application No. PCT/US2021/034895, filed May 28, 2021, which is incorporated by reference herein in its entirety.

### INCORPORATION BY REFERENCE OF SEQUENCE LISTING

The contents of the ASCII text file of the sequence listing named “PT1181_seqlist_ST25.txt” which is 210,428 bytes in size and created on Jul. 12, 2021 and electronically submitted via Patent Center are incorporated by reference herein in their entirety.

### BACKGROUND

Fermentation processes are used commercially at large scale to produce organic molecules such as ethanol, citric acid and lactic acid. In those processes, a carbohydrate is fed to an organism that is capable of metabolizing it to the desired fermentation product. The carbohydrate and organism are selected together so that the organism is capable of efficiently digesting the carbohydrate to form the product that is desired in good yield. It is becoming more common to use genetically engineered organisms in these processes, in order to optimize yields and process variables, or to enable particular carbohydrates to be metabolized.

### SUMMARY OF THE INVENTION

Various embodiments disclosed relate to a fermentation method for producing malonate. The fermentation method includes a) culturing a microorganism in the presence of a fermentation medium. The fermentation medium includes at least one carbon source; calcium ions ranging from 0.05 g/L to 1.6 g/L; and a base other than a calcium containing base. The fermentation method further comprises b) producing at least 40 grams/liter of malonate. Optionally, the malonate is produced at a rate of at least 0.3 g L⁻¹ h⁻¹ to 5 g L⁻¹ h⁻¹.

Various embodiments disclosed provide an engineered microorganism capable of producing malonate. The engineered microorganism includes a heterologous malonate-semialdehyde dehydrogenase. The heterologous malonate-semialdehyde dehydrogenase includes at least 90% sequence identity to SEQ ID No: 27. The engineered microorganism is capable of producing about at least 60 g/L at the end of a fermentation.

### DETAILED DESCRIPTION OF THE INVENTION

Reference will now be made in detail to various examples of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “0.1% to 5%” or “0.1% to 5%” should be interpreted to include not just 0.1% to 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” includes one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “substantially” means a majority of, or mostly, as in at least about 90%, preferably 95%, more preferably 99%, even more preferably 99.5, and even more preferably 99.99%, relative to a reference number. When used to indicate substantially zero, none, or free of, “substantially” as used herein means less than 1%, relative to a reference number.

In some modes of practice, the BLAST algorithm is used to compare and determine sequence similarity or identity. In addition, the presence or significance of gaps in the sequence which can be assigned a weight or score can be determined. These algorithms can also be used for determining nucleotide sequence similarity or identity. Parameters to determine relatedness are computed based on art known methods for calculating statistical similarity and the significance of the match determined. Gene products that are related are expected to have a high similarity, such as greater than 50% sequence identity. Exemplary parameters for determining relatedness of two or more sequences using the BLAST algorithm can be as follows.

Inspection of nucleic acid or amino acid sequences for two nucleic acids or two polypeptides will reveal sequence identity and similarities between the compared sequences. Sequence alignment and generation of sequence identity include global alignments and local alignments which are carried out using computational approaches. An alignment can be performed using BLAST (National Center for Biological Information (NCBI) Basic Local Alignment Search Tool) version 2.2.31 software with default parameters. Amino acid % sequence identity between amino acid sequences can be determined using standard protein BLAST with the following default parameters: Max target sequences: 100; Short queries: Automatically adjust parameters for short input sequences; Expect threshold: 10; Word size: 6; Max matches in a query range: 0; Matrix: BLOSUM62; Gap Costs: (Existence: 11, Extension: 1); Compositional adjustments: Conditional compositional score matrix adjustment; Filter: none selected; Mask: none selected. Nucleic acid % sequence identity between nucleic acid sequences can be determined using standard nucleotide BLAST with the following default parameters: Max target sequences: 100; Short queries: Automatically adjust parameters for short input sequences; Expect threshold: 10; Word size: 28; Max matches in a query range: 0; Match/Mismatch Scores: 1, −2; Gap costs: Linear; Filter: Low complexity regions; Mask: Mask for lookup table only. A sequence having an identity score of XX % (for example, 80%) with regard to a reference sequence using the NCBI BLAST version 2.2.31 algorithm with default parameters is considered to be at least XX % identical or, equivalently, have XX % sequence identity to the reference sequence.

In addition to the methods for recovering malonate from a fermentation broth, the methods described herein also include fermenting a carbohydrate using microbial cells at a suitable pH (as further described below) to produce the fermentation broth containing the malonate to be recovered.

As used herein, the term “fermentation broth” generally refers to a mixture derived from a microbial fermentation process, which contains a fermentation medium (liquid; comprising, for example, the produced malonate and other organic acids, salts, metals, residual sugars, and other fermentation by-products) and biomass (solid; comprising, for example, cells and cell debris).

As used herein, the term “fermentation” or “fermenting” generally refers to the feeding of a carbon source (e.g., a sugar, such as glucose) to a microorganism under conditions that enable the microorganism to use the carbon source to produce malonate.

As used herein, the term “malonate” or “malonate composition” includes compounds according to formulas I, II, and III shown below:

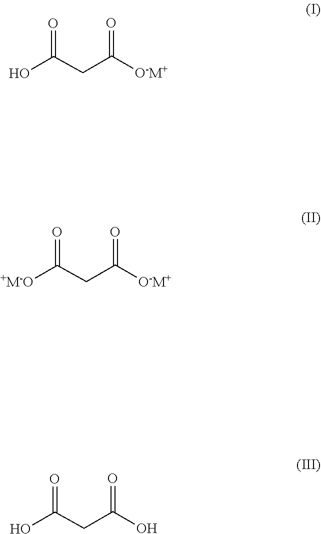

In formulas I and II, M is a Group I alkali metal cation (such as Li⁺, Na⁺, K⁺, ammonium (such as NH₄⁺), or a mixture, thereof. When referring to malonate concentration (e.g. malonate recovered) herein we are referring to the concentration of malonic acid equivalents (i.e. the sum of: monosalts of malonic acid (formula I), disalts of malonic acid (formula II), and free malonic acid (formula III) excluding metal ions and other cations.

In various examples, fermenting includes culturing a microorganism in the presence of at least one carbon source, allowing the microorganism to produce malonate for a period of time, and then isolating malonate produced by the microorganism. The fermentation method described herein is typically a batch or fed-batch fermentation method.

The carbon source may be any carbon source that is typically fermented by the provided microorganism. The carbon source may include maltodextrin, glucose, xylose, arabinose, sucrose, fructose, cellulose, glucose oligomers, glycerol, or a mixture thereof. In specific examples, the carbon source may include starch, a starch derivative, sucrose, or a glucose containing material. The glucose containing material, for example, may be a material containing at least 50 wt %, at least 60 wt %, at least 65 wt %, at least 70 wt %, at least 75 wt %, at least 80 wt %, at least 85 wt %, at least 90 wt %, at least 95 wt % glucose, and typically less than 99 wt % glucose.

Microorganisms that are typically used to produce malonate include a microorganism capable of producing malonic acid from fermentable carbon sources (e.g., glucose, sucrose, and/or other carbohydrates) include a yeast. An example of yeast is a Crabtree negative yeast. A specific example of a yeast includes a member of the Issatchenkia orientalis or Pichia fermentans clades. Issatchenkia orientalis (alternatively referred to as Candida krusei and Pichia kudriavzevii) is a preferred microorganism.

In some aspects, the microorganism may be an engineered microorganism that includes at least one genetic modification to a metabolic pathway associated with producing malonate. Examples of suitable genetic modifications include those to a polypeptide with malonate-semialdehyde dehydrogenase (MSADh) activity, meaning the ability to catalyze the conversion of malonate-semialdehyde to malonate and can be found in each of PCT/2020/066372, PCT/US2020/066390, and PCT/US2020/066411, the contents of each are hereby incorporated by reference. Other examples of suitable genetic modifications include those to a polypeptide with malonyl COA hydrolase activity, meaning the ability to catalyze the conversion of malonyl COA to malonate, an example of such a modification can be found in published PCT patent application WO 2021/042058 A2. Another example of suitable genetic modifications include: (i) a modification to a polypeptide with malonyl COA reductase activity, meaning the ability to catalyze the conversion of malonyl COA to malonate semialdehyde, an example of such a modifications can be found in published PCT patent application WO 2013/134424 A1; and (ii) a polypeptide with MSADh activity.

The malonate fermentation pathway genes in the yeast cells provided herein may be endogenous or heterologous. “Endogenous” as used herein refers to a genetic material such as a gene, a promoter and a terminator is “endogenous” to a cell if it is (i) native to the cell, (ii) present at the same location as that genetic material is present in the wild-type cell and (iii) under the regulatory control of its native promoter and its native terminator. The term “heterologous” means a molecule (e.g., polypeptide or nucleic acid) that is from a source that is different than the referenced organism or, where present, a referenced molecule. Accordingly, a gene or protein that is heterologous to a referenced organism is a gene or protein not found in the native form of that organism. For example, a specific glucoamylase (GA) gene found in a first fungal species and exogenously introduced into a second fungal species that is the host organism is “heterologous” to the second fungal organism. As another example, a specific glucoamylase gene from a fungal species that is modified from its native form with one or more nucleotide changes that affect the function of the gene is “heterologous”. An exogenous nucleic acid can be introduced into the host organism by well-known techniques and can be maintained external to the hosts chromosomal material (e.g., maintained on a non-integrating vector), or can be integrated into the host's chromosome, such as by a recombination event. An exogenous nucleic acid can encode an enzyme, or portion thereof, that is either homologous or heterologous to the host organism. All heterologous nucleic acids are also exogenous. For purposes of this application, genetic material such as genes, promoters and terminators is “exogenous” to a cell if it is (i) non-native to the cell and/or (ii) is native to the cell, but is present at a location different than where that genetic material is present in the wild-type cell and/or (iii) is under the regulatory control of a non-native promoter and/or non-native terminator. Extra copies of native genetic material are considered as “exogenous” for purposes of this invention, even if such extra copies are present at the same locus as that genetic material is present in the wild-type host strain. “Native” with regard to a metabolic pathway means a metabolic pathway that exists and is active in the wild-type host strain. Genetic material such as genes, promoters and terminators is “native” for purposes of this application if the genetic material has a sequence identical to (apart from individual-to-individual mutations which do not affect function) a genetic component that is present in the genome of the wild-type host cell (e.g., the exogenous genetic component is identical to an endogenous genetic component).”

An exogenous genetic component may have either a native or non-native sequence. An exogenous genetic component with a native sequence comprises a sequence identical to a genetic component that is present in the genome of a native cell (e.g., the exogenous genetic component is identical to an endogenous genetic component). However, the exogenous component is present at a different location in the host cell genome than the endogenous component. For example, an exogenous PYC gene that is identical to an endogenous PYC gene may be inserted into a yeast cell, resulting in a modified cell with a non-native (increased) number of PYC gene copies. An exogenous genetic component with a non-native sequence comprises a sequence that is not found in the genome of a native cell. For example, an exogenous PYC gene from a particular species may be inserted into a yeast cell of another species. An exogenous gene is integrated into the host cell genome in a functional manner, meaning that it is capable of producing an active protein in the host cell. However, in various examples the exogenous gene may be introduced into the cell as part of a vector that is stably maintained in the host cytoplasm. In other examples, the exogenous genetic component can be in a native location but can have a modification to its promoter or terminator.

In various examples, culturing of the cells provided herein to produce malonate may be divided up into phases. For example, the cell culture process may be divided into a cultivation phase, a production phase, and a recovery phase. One of ordinary skill in the art will recognize that the conditions used for these phases may be varied based on factors such as the species of microorganism being used.

The fermentation medium will typically contain nutrients as required by the particular microorganism, including a source of nitrogen (such as amino acids, proteins, inorganic nitrogen sources (such as ammonia or ammonium salts), and the like), and various vitamins, minerals, and the like. In some embodiments, the microorganisms of the invention is typically cultured in a chemically defined fermentation medium. In one example, the fermentation medium contains 0.05 g/L to 1.6 g/L of calcium ion (Ca²⁺) or 0.1 g/L to 1.2 g/L of Ca²⁺ or 0.1 g/L to 0.5 g/L Ca²⁺. The calcium ion is typically provided to the fermentation medium at the start of the fermentation or continuously during the course of fermentation. The calcium ion is typically provided to the fermentation medium in the form of a calcium containing compound. Examples of suitable calcium containing compounds include CaCO₃, CaCl₂, Ca(OH)₂, or a mixture thereof. Of the calcium containing compounds, Ca(OH)₂ is preferred. This is because, under some circumstances, CaCO₃ can be a slurry that tends to form sticky globs that can plug up piping in a fermentation system. Additionally, even though CaCl₂ functions well with most microorganisms, if a fermenter used in the fermentation is formed from stainless steel, Cl⁻ can contribute to causing corrosion in the fermenter. Ca(OH)₂ does not present any of the aforementioned drawbacks. For example, when Ca(OH)₂ is utilized, the typical concentration of Ca(OH)₂ that is added to the fermentation medium is in a range of 0.1 g/L to 3.2 g/L, or 0.18 g/L to 2.2 g/L, or 0.18 g/L to 0.92 g/L. Importantly, the calcium ions in the fermentation method do not function as a buffer.

Additional components of the fermentation medium typically can include 3-15 g/L ammonium sulfate (for example 3-7 g/L), 1-6 g/L phosphate anion (e.g. in the form of potassium dihydrogen phosphate), 0.25 g/L to 4 g/L (e.g. 0.4 to 0.6 g/L) magnesium sulfate, trace elements, vitamins and from about 90 to 180 g/L glucose (for example, from 130 to 170 g/L glucose). The pH may be allowed to range freely during cultivation, or may be buffered if necessary to prevent the pH from falling below or rising above predetermined levels. In various examples, the fermentation medium is inoculated with sufficient yeast cells that are the subject of the evaluation to produce an OD₆₀₀ of 1.0. Unless explicitly noted otherwise, OD₆₀₀ as used herein refers to an optical density measured at a wavelength of 600 nm with a 1 cm pathlength using a model DU600 spectrophotometer (Beckman Coulter). The cultivation temperature may range from 30-40° C., and the cultivation time may be up to 120 hours.

In one example, the concentration of cells in the fermentation medium is typically in the range of 0.1 to 20 g dry cells/liter of fermentation medium, from 0.1 to 5 g dry cells/liter of fermentation medium, or from 1 to 3 g dry cells/liter of fermentation medium during the production phase. The fermentation may be conducted aerobically or microaerobically, depending on pathway requirements. If desired, oxygen uptake rate (OUR) may be varied throughout fermentation as a process control (see, e.g., WO 03/102200). In some embodiments, the modified yeast cells provided herein are cultivated under conditions characterized by an oxygen uptake rate from 25 mmol L⁻¹ h⁻¹ to 60 mmol L⁻¹ h⁻¹, e.g., 30 mmol L⁻¹ h⁻¹ to 50 mmol L⁻¹ h⁻¹, of 35 mmol L⁻¹ h⁻¹ to 45 mmol L⁻¹ h⁻¹.

The fermentation medium may be buffered during the production phase such that the pH is maintained in a range of 2.5 to 6.2, for example from 2.5 to 5.0, from 2.5 to 4.5, from 3.0 to 4.3, from 3.0 to 4.0, and in some instances where a higher pH is desirable from 3.5 to 6.0, from 3.7 to 5.3. Suitable buffering agents are basic materials that neutralize the acid as it is formed, and include, for example, sodium hydroxide, potassium hydroxide, potassium carbonate, sodium carbonate, ammonium carbonate, ammonia, ammonium hydroxide and the like. The buffering agent does not include calcium. Preferably, potassium hydroxide is used as a buffering agent. In general, those buffering agents that have been used in conventional fermentation processes are also suitable here with the exception that buffering agents including calcium are not used.

Because calcium is not used as a buffer, the calcium ions in the fermentation medium, can be used as a feed source for the microorganism while any of the buffering agents described herein are used for pH control. The amount of buffering agent added is typically at a level sufficient to control the pH during malonate separation as described below. For example, the buffering agent may be present in a range of 0.8 wt % to 15 wt % of the fermentation medium or 16 wt % to 8 wt % of the fermentation medium. A molarity of the buffering agent is in a range of 0.15M to 3M or 0.5M to 1.5M.

In those embodiments where a buffered fermentation is utilized, acidic fermentation products may be neutralized to their corresponding salt as they are formed. In these embodiments, recovery of the acid involves regeneration of the free acid. This may be done by removing the cells and acidulating the fermentation broth with a strong acid such as sulfuric acid. This results in the formation of a salt by-product.

In other embodiments, the pH of the fermentation medium may be permitted to drop during cultivation from a starting pH that is at or above the pKa of malonic acid, typically 4.5 or higher, to at or below the pKa of the acid fermentation product, e.g., less than 4.5 or 4.0, such as in the range of 1.5 to 4.5, in the range of from 2.0 to 4.3, or in the range from 2.0 to 4.0.

In still other embodiments, fermentation may be carried out to produce a product acid by adjusting the pH of the fermentation broth to at or below the pKa of the product acid prior to or at the start of the fermentation process. The pH may thereafter be maintained at or below the pKa of the product acid throughout the cultivation. In various examples, the pH may be maintained at less than 4.5 or 4.0, such as in a range of 1.5 to 4.5, in a range of 2.0 to 4.3, or in a range of 2.0 to 4.0.

In various examples of the methods provided herein, the final yield of malonate from the carbon source is at least 10%, at least 20%, at least 30%, at least 40%, at least 50%, or greater than 50% of the theoretical yield. The concentration, or titer, of malonate will be a function of the yield as well as the starting concentration of the carbon source. In various examples, malonate is typically produced at a rate in a range of 0.3 g L⁻¹ h⁻¹ to 5 g L⁻¹ h⁻¹ or 0.8 g L⁻¹ h⁻¹ to 3 g L⁻¹ h⁻¹, or 1 g L⁻¹ h⁻¹ to 3 g L⁻¹ h⁻¹. In various examples, the titer may reach at least 40 g/L of malonate to 200 g/L or 60 g/L malonate to 140 g/L malonate over a time range of 15 hours to 200 hours or 20 hours to 100 hours.

According to various aspects the microorganism can be an engineered microorganism that is typically enhanced to produced elevated levels of malonate. According to various further aspects an engineered microorganism can produce less than 5 g/L of ethanol is produced after 36 hours. According to some further aspects the engineered microorganism can produce less than 3 g/L of glycerol, less than 1 g/L of glycerol, less than 0.5 g/L of glycerol at the time the fermentation medium is harvested.

Once produced, any method known in the art can be used to isolate malonate from the fermentation medium. For example, common separation techniques can be used to remove the biomass from the fermentation broth, and common isolation procedures (e.g., extraction, distillation, and ion-exchange procedures) can be used to obtain the malonate from the microorganism-free fermentation broth. In addition, malonate can be isolated while it is being produced, or it can be isolated from the fermentation broth after the product production phase has been terminated. The pH used during fermentation can be helpful for a subsequent isolation step as described below.

A Group I alkali metal cation and/or ammonium cation used in the recovery of the malonate and is typically added after the fermentation, or at least some or all of the required Group I alkali metal cation and/or ammonium cation is typically added during the fermentation. For example, the fermentation broth can include a source of the Group I alkali metal cation, such as a Group I alkali metal carbonate, Group I alkali metal bicarbonate, Group I alkali metal oxide or a Group I alkali metal hydroxide for pH control during the fermentation. The Group I alkali metal carbonate, Group I alkali metal bicarbonate, Group I alkali metal oxide or a Group I alkali metal hydroxide can help maintain the pH of the fermentation broth at the levels described above. A source of ammonium cations such as ammonia, ammonium hydroxide, ammonium bicarbonate, ammonium carbonate and the like is typically added as a source of nitrogen for the fermentation and to adjust or maintain the pH during the fermentation.

As used herein, the term “Group I alkali metal cations” generally means, for example, cations of lithium (Li), sodium (Na), and potassium (K); that is to Li⁺, Na⁺, and K⁺. Further, as used herein, the term “ammonium cation” includes any cation of the formula R₄N⁺, wherein R is hydrogen or alkyl. If potassium hydroxide is used as a base during fermentation, the potassium ion used in fermentation is typically included at such a level that is high enough to raise the pH of the fermentation broth and be useful for isolation.

Suitable sources of the Group I alkali metal cations include, but are not limited to, sodium hydroxide, potassium hydroxide, potassium carbonate, sodium carbonate, and the like. Suitable sources of ammonium cation include, for example, ammonium hydroxide, ammonium carbonate, and the like.

The source of the Group I alkali metal cation can also be from compounds that do not necessarily control the pH of the fermentation broth or the aqueous solution used for recovering the malonate described herein. For example, the Group I alkali metal cation source can be from compounds such as, sodium chloride, potassium chloride, ammonium chloride, and the like. Regardless of the purpose (e.g., pH control) or properties, the Group I alkali metal cation source is typically added to the fermentation broth during or at the end of the fermentation, or to the aqueous solution used for recovering the malonate described herein at any time prior to or during the recovery of the malonate.

Removing water from the aqueous solutions (e.g. the fermentation broth and/or the aqueous filtrate) is typically performed using any suitable method at any suitable temperature and pressure. In addition, the removing water from the fermentation broth to increase the concentration of the malonate and produce an aqueous concentrate can be implemented two or more times as described in greater detail herein. For example, the water can be removed at less than atmospheric pressure, such as at less than 101.325 kPa. In addition, or alternatively, the water typically can be removed at a temperature (e.g., an aqueous bulk temperature) of 100° C. or less, 90° C. or less, 80° C. or less, preferably 70° C. or less, more preferably 60° C. or less, 50° C. or less; from 40° C. to 100° C., from 50° C. to 90° C., from 50° C. to 80° C., from 50° C. to 70° C. In order to minimize the premature crystallization/precipitation of the malonate, the temperature of the solution containing the malonate is typically maintained at or above 30° C., at least 35° C., at least 40° C., at least 50° C., at least 60° C.; from 30° C. to 100° C., 30° C. to 90° C. or 40° C. to 80° C. if the concentration of the malonate is at least 400 g/kg; and preferably, if 500 g/kg or greater malonate concentration, at least 40° C., at least 45° C. The temperature of the solution containing the malonate preferably is kept lower than 90° C., preferably less than 80° C., and less than 70° C. to minimize decarboxylation of the malonate.

Although a lower limit of 400 g/kg malonate is described in connection with the removing of water from the aqueous solution obtained from the fermentation broth comprising the malonate to increase the concentration of the malonate, the methods described herein also include removing water from the fermentation broth and the aqueous filtrate comprising the malonate to enhance the recovery of the malonate include increasing the concentration of the malonate to at least 400 g/kg; at least 425 g/kg, at least 450 g/kg; at least 475 g/kg, at least 500 g/kg, at least 525 g/kg, at least 550 g/kg, at least 575 g/kg, at least 600 g/kg; less than 800 g/kg, less than 775 g/kg, such as from 400 g/kg to 775 g/kg, from 450 g/kg to 700 g/kg, and from 500 g/kg to 600 g/kg.

The pH of the aqueous concentrate and/or the aqueous filtrate during the recovery of the malonate is typically from 2.5 to 5.0, more preferably from 2.5 to 4.5, from 3.0 to 4.3 (for example from 3.0 to 4.0).

Typically, concentration of the malonate after a cell removal step is between 30 g/kg and 775 g/kg the malonate. Preferably, the cells are removed and the concentration of the malonate after removal step typically is from 30 g/kg to 400 g/kg, 50 g/kg to 350 g/kg, 80 g/kg to 350 g/kg. Or the cells are removed and the concentration of the malonate after removal step typically is 400 g/kg to 800 g/kg, 400 g/kg to 700 g/kg, 500 g/kg to 600 g/kg. For example, the biomass is typically removed using one or more filtration steps or one or more centrifugation steps. Centrifugation is typically carried out in a decanter centrifuge, such as a horizontal-type decanter centrifuge, a disc stack centrifuge, or hydrocyclones.

As mentioned herein, removing water from the aqueous solution to increase the concentration of the malonate and produce an aqueous concentrate can be implemented two or more times. For example, removing water can comprise removing water from the fermentation broth and can further comprise removing water to provide a first aqueous concentrate.

The malonate is typically recovered from a first aqueous concentrate and the second aqueous concentrate by any suitable method including precipitating or crystallizing. The step of recovering the malonate typically includes lowering the temperature of the first aqueous concentrate and/or the second aqueous concentrate to below 40° C., below 35° C., below 30° C., below 25° C., below 20° C., below 15° C., below 10° C., below 5° C., below 0° C.; typically from 0° C. to 30° C., from 4° C. to 25° C. Those of skill in the art will recognize that the temperature at which the malonate can effectively be recovered, will depend on the concentration of the malonate in first aqueous concentrate and/or second aqueous concentrate, the pH of the relevant first aqueous concentrate and/or second aqueous concentrate, the number of recovery steps that are utilized, and the desired overall yield of the malonate resulting from the recovery steps. For example, the malonate can be recovered at a higher temperature if the relevant aqueous concentrate comprises 700 g/kg of the compound of the malonate relative to when the concentration of the malonate is 400 g/kg in the aqueous concentrate. However, where a higher yield of the malonate is desirable, a lower temperature (typically at or below 30° C. is utilized).

The malonate recovered is typically a mixture of malonic acid, a compound of formula I, and potentially a compound of formula II. For example, for pHs during the recovery of the first crop at a pH between 2.5 and 5.0 (preferably from 2.5 and 4.5, for example from 3.0 to 4.5, from 3.0 to 4.3), and with a second crop recovered without adjusting the pH (i.e. the malonate in the filtrate is concentrated, but no base or acid is added to adjust the pH), malonate recovered from the first aqueous concentrate typically comprises: at least 40 wt % (for example, at least 50 wt %, at least 55 wt %, at least 60 wt % of the compound of formula 1 (for example from 55 wt % to 75 wt % (from 60 wt % to 75 wt %) of the compound of formula 1; less than 50 wt % (for example, less than 45 wt %, less than 40 wt % (from 1 wt % to 45 wt %, from 2 wt % to 40 wt %, from 20 wt % to 40 wt %)) of malonic acid; and less than 25 wt % (for example, less than 15 wt %, less than 10 wt %, less than 5 wt %, less than 2 wt %, less than 1 wt % (for example from 0.1 wt % to 10 wt %) of the compound of formula II. malonate recovered from the second aqueous concentrate typically comprises: at least 40 wt % (for example, at least 45 wt %, at least 60 wt % (for example from 40 wt % to 90 wt %, from 60 wt % to 90 wt %) of the compound of formula 1; less than 55 wt % (for example, less than 50 wt %, less than 15 wt % for example, from 1 wt % to 55 wt %, from 1 wt % to 15 wt %) of malonic acid; and less than 25 wt % (for example, less than 20 wt %, less than 17 wt % (for example from 0.05 wt % to 20 wt %, from 1 wt % to 17 wt %)) of the compound of formula II. Typically the total the malonate recovered (for example from the sum of the first and the second crop comprises: at least 40 percent (for example at least 50 percent, at least 60 percent) by weight of the compound of formula I; no greater than 25 percent (for example no greater than 20 percent, no greater than 15 percent, no greater than 10 percent, or not greater than 5 percent) by weight of the compound of formula II; and less than 50 percent (less than 45 percent, less than 40 percent, less than 35 percent, less than 30 percent) by weight malonic acid.

Table 1, below provides an example of the typical malonate compositions recovered through the use of the methods described herein:

TABLE 1

| | | | | | | | | | Composite | | | |
|---|---|---|---|---|---|---|---|---|---|---|---|---|
| | 1st Crop | | | | 2nd Crop | | | | (i.e. 1^{st} + 2^{nd} Crops) | | | |
| | % | % Mono- | % Dipo- | | % | % Mono- | % Dipo- | | % | % Mono- | % Dipo- | |
| | Malonic | potassium | tassium | | Malonic | potassium | tassium | Recovery | Malonic | potassium | tassium | Total |
| pH of 1^{st} | Acid | Malonate | Malonate | Recovery | Acid | Malonate | Malonate | Yield of | Acid | Malonate | Malonate | Malonate |
| crop | recov- | recov- | recov- | Yield of | recov- | recov- | recov- | Second | recov- | recov- | recov- | Recov- |
| recovery | ered | ered | ered | 1st Crop¹ | ered | ered | ered | Crop² | ered | ered | ered | ered³ |
| 2.5 | 41.3% | 58.4% | 0.3% | 69.2% | 52.8% | 47.1% | 0.1% | 69.8% | 44.0% | 55.7% | 0.3% | 90.7% |
| 3 | 36.9% | 62.7% | 0.4% | 68.5% | 11.6% | 86.2% | 2.1% | 90.3% | 29.5% | 69.6% | 0.9% | 96.9% |
| 3.5 | 28.3% | 71.1% | 0.6% | 70.9% | 1.5% | 83.3% | 15.2% | 82.7% | 21.5% | 74.2% | 4.3% | 95.0% |
| 4 | 2.8% | 88.0% | 9.2% | 70.9% | 1.6% | 83.8% | 14.6% | 78.8% | 2.5% | 87.0% | 10.5% | 93.8% |

¹Yield based on total recovery of initial malonate
²Yield based on malonate recovery from 1st crop filtrate
³Yield based on malonate recovery in 1st and 2nd crops

The pH of the first aqueous concentrate during the recovery of the first crop typically is as described above. Typically, the pH of the second aqueous concentrate during the recovery of the second crop is not adjusted by the addition of an acid or a base. The pH of the second aqueous concentrate during the recovery of the second crop is typically 0.2 to 1.0 pH unit higher than the pH utilized in the recovery of the first crop, more typically a pH during the second crop that is 0.2 to 0.6 higher than the pH utilized during the recovery of the first crop.

Lowering the temperature of the first aqueous concentrate (and the second aqueous concentrate if a second crop is recovered) causes the precipitation of the malonate from the first aqueous concentrate (and the second aqueous concentrate if a second crop is recovered). The malonate can then be removed by, e.g., filtration, to give an aqueous filtrate comprising the malonate and a first crop of precipitate of recovered the malonate. The aqueous filtrate comprising the malonate can, in turn, be concentrated by removing water, as described herein, from the aqueous filtrate to increase the malonate concentration typically to a concentration of from 380 g/kg to 775 g/kg (preferably from 400 g/kg to 700 g/kg, for example from 500 g/kg to 600 g/kg malonate concentration) to form a second aqueous concentrate.

Removing water to form the second aqueous concentrate is typically performed using any suitable method at any suitable temperature and pressure. Preferably, the temperatures and pressured described for use in removing water to form the first aqueous concentrate are utilized.

A second crop of precipitate of the malonate typically is recovered from the second aqueous concentrate by lowering the temperature of the second aqueous concentrate to similar temperatures as described above for use in recovering the first crop of the malonate. The sum of the amounts of the malonate recovered in the first crop plus the second crop typically constitute at least 70%, at least 75%, at least 80%, at least 85%, at least 90%, at least 95%; 85% to 99%, 85% to 98% or 90% to 99% of the malonate present in the fermentation broth. If no solvent assisted precipitation is used during recovery of the first crop or second crop, the recovery of the malonate typically is from 70% to 90% of the malonate present in the fermentation broth. The higher recoveries (e.g. greater than 85%, at least 90%, for example from 90% to 98%, from 93% to 98% of the malonate in the fermentation broth recovered) typically are achieved using solvent assisted precipitation during the first crop and/or second crop recovery.

It is contemplated that, a third, fourth or even a fifth crop (or more) of precipitate of the malonate can be obtained by continuing to remove water from additional aqueous filtrates that contain the malonate by concentration of the malonate, and lowering the temperatures as set forth for the first crop and second crop of the malonate precipitate. As mentioned earlier, the precipitation from during any crop may include crystallization or precipitation by other means known to one of ordinary skill in the art.

As mentioned above, the recovery of the compound of the formula I from any of the first aqueous concentrate and/or the second aqueous concentrate (if present) can, in addition to or instead of lowering the temperature, be accomplished by adding an organic solvent. For example, the methods described herein can include adding an organic solvent to the second aqueous concentrate to enhance the recovery of the malonate. Preferably, the organic solvent is water-miscible. As mentioned above, preferably the temperature of the aqueous concentrate is reduced to less than 40° C. to enhance the recovery of the malonate. Preferably, a water-miscible organic solvent is added to the second aqueous concentrate to enhance the recovery of the malonate from the second aqueous concentrate. In a particularly preferred aspect, a water-miscible organic solvent is added to the second aqueous concentrate, but is not added to the first aqueous concentrate. Suitable water-miscible organic solvents include, but are not limited to alcohols (e.g., ethanol), ketones (e.g., acetone), nitriles (e.g., acetonitrile), ethers (e.g., tetrahydrofuran), and combinations thereof. Suitable alcohols include C₁-C₅ alcohols (preferably C₁-C₃), such as methanol, ethanol, propanol, isopropanol, and the like, and combinations thereof. In some examples, ethanol is the water-miscible organic solvent. In some instances, the water-miscible organic solvent is added prior to recovering the malonate from the first and/or second aqueous concentrate.

Those of skill in the art will recognize that the recovered malonate can be subsequently transformed into a purer form of malonic acid or into a mono- or di-ester of malonic acid of the formula IV:

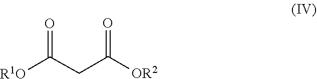

wherein R¹ and R² can be the same or different and can be H, C₁-C₁₀ alkyl (e.g., preferred is C₁-C₆ and C₂-C₆), which is optionally substituted with halo (e.g., fluoro, chloro or bromo), C₄-C₁₀ aryl (e.g., phenyl or naphthyl) or C₁-C₅ alkoxy, provided that R¹ and R² are not both H at the same time.

Malonate produced using the methods disclosed herein can be chemically converted into other organic compounds. For example, malonate can be hydrogenated to form 1,3 propanediol, a valuable polyester monomer. Propanediol also can be created from malonate using polypeptides having oxidoreductase activity in vitro or in vivo. Hydrogenating an organic acid such as malonic acid can be performed using any method such as those used to hydrogenate succinic acid and/or lactic acid. For example, malonic acid can be hydrogenated using a metal catalyst. In another example malonic acid to form acrylic acid using any known method for performing dehydration reactions. For example, malonic acid can be heated in the presence of a catalyst (e.g., a metal or mineral acid catalyst) to form acrylic acid.

### EXAMPLES

Various embodiments of the present invention can be better understood by reference to the following Examples which are offered by way of illustration. The present invention is not limited to the Examples given herein.

### Example 1: Construction of Strains of Pichia kudriavzevii for the Production of Malonate

### Strain 1

Strain 1 is Strain C in WO2017/024150 (the contents of which are hereby incorporated by reference), which is deleted for both alleles of the URA3 gene, making the strain unable to grown on media that does not contain uracil.

### Strains 2 through 11

Strains 2 through 11 are constructed by transforming the Parent Strain in Table 2 with the Seq ID Nos. from the same row in Table 2, resulting in the Constructed Strain shown in Table 2. For example, Strain 1 is simultaneously transformed with Seq ID No. 1 and Seq ID No. 2, resulting in Constructed Strain 2. Subsequently, Strain 2 is simultaneously transformed with Seq ID No. 2 and Seq ID No. 3, resulting in Constructed Strain 3. Transformants are selected on the appropriate media for the Marker listed in Table 2. Transformants bearing the URA marker are selected on ScD-Ura media. Transformants bearing the HYGRO marker are selected on YPD media containing hygromycin (YPD+Hygro300). Transformants bearing the MEL marker are selected on YNB+Melibiose+X-α-gal solid media. All transformants are verified by PCR.

If the marker to be used, as described in the Marker column of Table 2, is already present in the Parent Strain, Seq ID No. 21 will first need to be transformed into the Parent Strain to remove the marker. SEQ ID NO: 21 contains the following elements: i) a Cre recombinase expressed by the native PDC1 promoter, ii) an expression cassette containing the ScSUC2 expressed by the native PGK1 promoter, and iii) an autonomously replicating sequence (ARS). Transformants are selected on YNB+20 g/L Sucrose+X-α-gal media. Resulting transformants are streaked for single colony isolation on YPD+X-α-gal media. A single white colony is selected, and correct recycling of marker is verified by polyermase chain reaction (PCR) procedures.

TABLE 2

| Parent | | | Constructed | | Integration |
|---|---|---|---|---|---|
| Strain | SeqID A | SeqID B | Strain | Marker | Locus |
| Strain 1 | SeqID No. | SeqID No. | Strain 2 | URA | mdhB |
| | 1 | 2 | | | |
| Strain 2 | SeqID No. | SeqID No. | Strain 3 | HYGRO | mdhB |
| | 3 | 4 | | | |
| Strain 3 | SeqID No. | SeqID No. | Strain 4 | URA | YMR226c |
| | 5 | 6 | | | |
| Strain 4 | SeqID No. | SeqID No. | Strain 5 | MEL | PDC |
| | 7 | 8 | | | |
| Strain 5 | SeqID No. | SeqID No. | Strain 6 | URA | PDC |
| | 9 | 10 | | | |
| Strain 6 | SeqID No. | SeqID No. | Strain 7 | HYGRO | YMR226c |
| | 11 | 12 | | | |
| Strain 7 | SeqID No. | SeqID No. | Strain 8 | URA | YMR226c |
| | 13 | 14 | | | |
| Strain 8 | SeqID No. | SeqID No. | Strain 9 | URA | YMR226c |
| | 15 | 16 | | | |
| Strain 9 | SeqID No. | SeqID No. | Strain 10 | URA | GPD |
| | 17 | 18 | | | |
| Strain 10 | SeqID No. | SeqID No. | Strain 11 | HYGRO | GPD |
| | 19 | 20 | | | |

TABLE 3
Table 3 lists the Seq ID No. used for each protein that
are integrated to produce malonate in the strain.

| Seq ID No | Protein |
|---|---|
| Seq ID No. 22 | ADC |
| Seq ID No. 23 | PYC |
| Seq ID No. 24 | AAT |
| Seq ID No. 25 | PYD |
| Seq ID No. 26 | MSADh |

TABLE 4
SCD-Ura Plates

| | |
|---|---|
| Difco ™ Yeast Nitrogen Base without amino | 6.7 g |
| acids (BD #291940) | |
| Glucose | 20 g |
| Agar | 20 g |
| SC-Ura Mixture (MP Biomedicals #4410- | 2 g |
| 622) | |
| Distilled H₂O | to 1 L |

Autoclave at 110° C. for 25 min

### Example 2: Improved Malonate Production in Fermentation from the Addition of 0.36 g/L Ca²⁺ in the Form of 1 g/L CaCl₂

Fermentation of Strain 9 is carried out in Sartorius Ambr250 automated bioreactor system. The working volume is 200 mL. Seed media and fermentation media are filter sterilized through a 0.2 μm filter. The inoculum includes a two stage shake flask seed. The first stage seed includes 250 mL baffled Erlenmeyer shake flasks containing 25 mL sterile seed media (composition listed in Table 5). A sterile YPD plate [1% Yeast extract, 2% Peptone, 2% Glucose, 2% Agar] is streaked with Strain 2.10 and incubated at room temperature for 3 days. A slurry is made by dispensing a loop full of solid culture from the YPD plate into 5 mL of sterile seed media. The slurry is used to inoculate the first stage seed shake flasks. The first stage seed flasks are incubated at 30° C. at 300 RPM and 70% humidity in an Infors Multitron shaking incubator with a 2.5 cm throw (model AJ125C) for 16-20 hours. The second stage seed includes 250 mL baffled Erlenmeyer shake flasks containing 25 mL sterile seed media and is inoculated with a volume of culture from first stage seed. The volume of first stage seed culture is selected such that the starting cell density in a second stage seed flask is between 0.27 g/L and 0.54 g/L cell dry weight (CDW). The second stage seed shake flasks are incubated at 34° C. and 300 RPM and 70% humidity in an Infors Multitron shaking incubator with a 2.5 cm throw (model AJ125C) for 4 hours. The culture from the second stage shake flask is harvested when the cell density of the biomass is in the range of 2.2-4.4 g/L CDW. Based on the cell density, a volume of the harvested culture is used to inoculate the sterile fermentation media in the Ambr250 bioreactors to arrive at an initial cell density of 0.03-0.04 g/L CDW in the bioreactor. The fermentation media composition is listed in Table 9. The fermentation process is run at a temperature of 34° C. The fermenters are sparged with air at a flow rate of 100 standard mL/min and agitation is set to 1850 rpm. The pH is controlled at 4.13 using 300 g/L KOH base. The process is run in a simultaneous saccharification and fermentation mode with maltodextrin as the carbon source and aminoglycosidase from Aspergillus niger (Sigma A7095) as the saccharification enzyme. 0.025 μL of aminoglycosidase per liter media is added immediately prior to inoculation. The fermentation is operated such that after a desired cell density is attained, dissolved oxygen (DO) limitation is achieved, and the DO remains at less than 2% of atmospheric air saturation for at least the following 10 hours. The onset of dissolved oxygen limitation marks the beginning of the production phase. The total fermentation time is roughly 146 hours, while the production time is 125 h. The malonate production metrics during this fermentation are presented in Table 10.

TABLE 5
Seed media composition

| | | | Vendor and |
|---|---|---|---|
| Chemical | Concentration | | Catalogue number |
| Glucose | 50.0 | g/L | Fisher# D16-1 |
| Glycerol (USP Grade) | 0.375 | g/L | Fisher# G33-1 |
| 2-ethane sulfonic acid (MES) | 13.7 | g/L | Sigma #M8250 |
| buffer | | | |
| 25x DMu salts: Table 6. | 40 | mL/L | N/A |
| 1000x DM1 Full Vitamin Solution: | 1 | mL/L | N/A |
| Table 7 | | | |
| 1000x DM1 TE Solution: Table 8 | 1 | mL/L | N/A |

TABLE 6
25X DMu salts

| | | Vendor and |
|---|---|---|
| Chemical | g/L @ 25X | Catalogue number |
| Urea | 57.0 | MP# 821530 |
| KH₂PO₄ | 75.0 | Fisher# P285-3 |
| MgSO₄*7H₂O | 12.5 | Fisher# M63-500 |
| Deionized water | Volume to | N/A |
| | 1 L | |

TABLE 7
1000X DM1 Full Vitamin solution

| | | Vendor and |
|---|---|---|
| Chemical | g/L | Catalogue number |
| Biotin (D−) | 0.05 | Acros# 230090250 |
| Ca D(+) pantothenate | 1.00 | Sigma# C8731 |
| Nicotinic acid | 5.00 | Sigma# N1426 |
| Myo-inositol | 25.00 | Acros# 122265000 |
| Thiamine hydrochloride | 1.00 | Fisher# O4700 |
| Pyridoxine hydrochloride | 1.00 | Fisher# BP2677 |
| p-aminobenzoic acid | 0.20 | Sigma# A9878 |

TABLE 8
1000X DM1 TE solution

| | | Vendor and |
|---|---|---|
| Chemical | g/L | Catalogue number |
| C₁₀H₁₄N₂Na₂O₈•2H₂O | 15.00 | Fisher# BP120 |
| ZnSO₄•7H₂O | 4.50 | Sigma# Z4570 |
| MnCl₂•2H₂O | 1.24 | Emsure# A1006034645 |
| CoCl₂•6H₂O | 0.30 | Sigma# C8661 |
| CuSO₄•5H₂O | 0.30 | Sigma# C7631 |
| Na₂MoO₄•2H₂O | 0.40 | Sigma# M1003 |
| CaCl₂•2H₂O | 4.50 | EMD Millipore# 208290 |
| FeSO₄•7H₂O | 3.00 | Sigma# 215422 |
| H₃BO₃ | 1.00 | Spectrum# 134924 |
| KI | 0.10 | Sigma# P2963 |

TABLE 9
Fermentation media composition

| | Concentration | Vendor and |
|---|---|---|
| Chemical | (g/kg) | Catalogue number |
| Maltodextrin | 100 | Aldrich# 419680 |
| Glycerol | 0.1 | Fisher# G33-1 |
| Lubrizol Antifoam (1:100 | 0.2 | N/A |
| diluted) | | |
| APK | 3.5 | N/A |
| Urea (49.4% solution) | 2.9 | MP# 821530 |
| MgSO₄•7H₂O | 0.25 | Fisher# M63-500 |
| 1000x Vitamin Solution | 1 | N/A |
| 100x Trace Solution | 1 | N/A |

The Ambr250 system records real time measurements of temperature, airflow, agitation, pH, dissolved oxygen, and off-gas composition. Oxygen uptake rate (OUR) and carbon dioxide exchange rate (CER) are calculated based on off-gas analysis.

Optical density is measured at 600 nm with a 1 cm pathlength using a spectrophotometer (Thermo Scientific). Cell concentration is obtained from optical density measurement using a conversion factor that was established based on previous experimentation.

Samples are obtained at several time points during the course of the fermentation. These samples are used for optical density (600 nm) measurement and analyzed using high performance liquid chromatography (HPLC).

Malonate titer results from the fermentation described above is labeled as a “reference” condition in in Table 10. The process is repeated in a similar manner with the difference that 1.00 g/L CaCl₂ is added to the fermentation media, malonate titer results from this process are labeled as “addition of 1 g/L CaCl₂” in Table 10.

TABLE 10
Fermentation metrics for malonate production
using Strain 2.10 in Ambr250 bioreactor

| | Malonate titer (g/L) at | |
|---|---|---|
| | end of fermentation | Malonate |
| | (t = 146 hours after | productivity |
| Condition | inoculation) | (g L¹ h¹) |
| Reference | 48.6 | 0.33 |
| Addition of 1 g/L CaCl₂ | 56.5 | 0.39 |

As shown in Table 10, the addition of 0.36 g/L Ca²⁺ in the form of 1.00 g/L CaCl₂ resulted in an improvement in the rate and final titer of malonate in a fermentation with a malonate producing yeast. A

### Example 3: Fermentations with Strains Deleted in Both of Both Copies of GPD (glycerol 3-phosphate dehydrogenase)

The fermentations of Example 2 are repeated with the addition of the 1/g CaCl₂ added to the fermentation media in the bioreactors just prior to inoculation. The fermentations are repeated with the following 2 strains: Strain 9, and Strain 11. Strain 11 is a derivative of strain 9 and is deleted in both alleles of the GPD gene. Strain 9 produces more than 8 g/L glycerol, which is subsequently reconsumed. With strain 11, the glycerol level never exceeds 0.5 g/L.

The results show that GPD deletion in a yeast allows for a more robust and flexible fermentation process with respect to avoiding glycerol production. In a fermentation process with a GPD deleted yeast, the fermentation broth can be harvested at any point without risk of residual glycerol being at an unacceptable level (e.g., greater than 1 g/L). In a yeast with intact GPD genes, if an acceptably low level of residual glycerol (i.e., less than 2.5 g/L) is to be avoided, the broth can only be harvested after glycerol reconsumption is complete.

As a byproduct, glycerol represents not only a yield loss (of malonate produced), but can also be problematic in downstream processing steps, as the glycerol will react with organic acids (such as malonic acid) and salts thereof to form esters under conditions where water is minimized.

### Example 4: Further Improved Strain

A strain similar to Strain 1 is modified to restore the URA gene. The modified strain is evolved in a Chemostat for three months in the presence of malonate to select for strains with malonate tolerance (e.g., highest growth rate in the presence of malonate). The strain with the highest malonate tolerance is selected for further engineering. The engineering includes deleting all copies of URA genes. This is followed by carrying out steps similar to those set forth in Example 1, which produces a strain similar to Strain 9 except for having a higher copy number of a different MSADh (identified as SEQ ID 27) and a higher copy number of ADC (identified as SEQ ID 22). The engineered strain produces malonate having a production of 0.8 g L⁻¹ h⁻¹ and a titer of 70 g/L (t=88 hrs) and a 38% yield malonate from glucose.

### EXEMPLARY EMBODIMENTS

The following exemplary embodiments are provided, the numbering of which is not to be construed as designating levels of importance:

Aspect 1 provides a fermentation method for producing malonate, the method comprising:
a) culturing a microorganism in the presence of a fermentation medium, the fermentation medium comprising:
   at least one carbon source;
   calcium ions ranging from 0.05 g/L to 1.6 g/L; and
   a base other than a calcium containing base; and
b) producing at least 40 grams/liter of malonate (for example, in a range of from 40 grams/liter to 200 grams/liter, from 60 grams/liter to 150 grams/liter, from 80 grams/liter to 150 grams/liter, from 90 grams/liter to 150 grams/liter, or from 100 grams/liter to 150 grams/liter malonate), wherein optionally, the malonate is produced at a rate of at least 0.3 g L⁻¹ h⁻¹ to 5 g L⁻¹ h⁻¹.

Aspect 2 provides the fermentation method of Aspect 1, wherein the carbon source comprises maltodextrin, glucose, xylose, arabinose, sucrose, fructose, cellulose, glucose oligomers, glycerol, or a mixture thereof.

Aspect 3 provides the fermentation method of any one of Aspects 1 or 2, wherein the carbon source comprises starch, a starch derivative, sucrose, a glucose containing material, for example, a material containing at least 50 wt %, at least 60 wt %, at least 65 wt %, at least 70 wt %, at least 75 wt %, at least 80 wt %, at least 85 wt %, at least 90 wt %, at least 95 wt % glucose, and typically less than 99 wt % glucose (for example, less than 98 wt % glucose), or mixture thereof.

Aspect 4 provides the fermentation method of any one of Aspects 1-3, wherein the calcium ions are provided from a calcium ion containing compound that is added to the fermentation medium.

Aspect 5 provides the fermentation method of Aspect 4, wherein the calcium ion containing compound comprises CaCO₃, CaCl₂, Ca(OH)₂, or a mixture thereof.

Aspect 6 provides the fermentation method of any one of Aspects 4 or 5, wherein the calcium ion containing compound comprises Ca(OH)₂.

Aspect 7 provides the fermentation method of any one of Aspects 4-6, wherein the calcium ion is provided to the fermentation medium at a concentration in a range of 0.1 g/L to 1.2 g/L.

Aspect 8 provides the fermentation method of any one of Aspects 4-7, wherein the calcium ion is provided to the fermentation medium at a concentration in a range of 0.1 g/L to 0.5 g/L.

Aspect 9 provides the fermentation method of any one of Aspects 1-8, wherein

the calcium ions are present at a start of the fermentation method.

Aspect 10 provides the fermentation method of any one of Aspects 1-9, wherein the calcium ions are added during the fermentation method.

Aspect 11 provides the fermentation method of any one of Aspects 1-10, wherein an oxygen uptake rate of the microorganism during the fermentation method is in a range of from 20 mmol L⁻¹ h⁻¹ to 60 mmol L⁻¹ h⁻¹.

Aspect 12 provides the fermentation method of any one of Aspects 1-11, wherein an oxygen uptake rate of the microorganism during the fermentation method is in a range of from 30 mmol L⁻¹ h⁻¹ to 50 mmol L⁻¹ h⁻¹, for example from 35 mmol L⁻¹ h⁻¹ to 50 mmol L⁻¹ h⁻¹.

Aspect 13 provides the fermentation method of any one of Aspects 1-12, wherein the base comprises KOH, NaOH, LiOH, and ammonia, or a mixture thereof.

Aspect 14 provides the fermentation method of any one of Aspects 1-13, wherein the base comprises KOH.

Aspect 15 provides the fermentation method of any one of Aspects 1-14, wherein the base is in a range of from 0.8 wt % to 15 wt % of the fermentation medium.

Aspect 16 provides the fermentation method of any one of Aspects 1-15, wherein the base is in a range of from 1.6 wt % to 8 wt % of the fermentation medium.

Aspect 17 provides the fermentation method of any one of Aspects 1-16, wherein a pH during fermentation is in a range of 2.5 to 7.

Aspect 18 provides the fermentation method of any one of Aspects 1-17, wherein a pH during fermentation is in a range of 2.5 to 6.2, example from 2.5 to 5.0, from 2.5 to 4.5, from 3.0 to 4.0, from 3.0 to 3.5, and in some instances where a higher pH is desirable from 3.5 to 6.0, from 3.7 to 5.3.

Aspect 19 provides the fermentation method of any one of Aspects 1-18, wherein the base functions to control a pH of the fermentation medium.

Aspect 20 provides the fermentation method of any one of Aspects 1-19, wherein the fermentation method is a batch or fed-batch fermentation process.

Aspect 21 provides the fermentation method of any one of Aspects 1-20, wherein the microorganism comprises an engineered microorganism.

Aspect 22 provides the fermentation method of any one of Aspects 1-21, wherein the microorganism comprises yeast, for example, a genetically modified Crabtree negative yeast.

Aspect 23 provides the fermentation method of Aspect 22, wherein the yeast comprises at least one of the Issatchenkia orientalis or Pichia fermentans clades, preferably Issatchenkia orientalis.

Aspect 24 provides the fermentation method of Aspect 23, wherein less than 3 g/L glycerol is present in a fermentation broth comprising the fermentation medium at the time the broth is harvested.

Aspect 25 provides the fermentation method of Aspect 24, wherein less than 1 g/L glycerol is present in the fermentation broth at the time the broth is harvested.

Aspect 26 provides the fermentation method of any one of Aspects 1-25, wherein the fermentation method is capable of producing 40 g/L to 200 g/L of malonate over a time in a range of 15 hours to 200 hours.

Aspect 27 provides the fermentation method of any one of Aspects 1-26, wherein the fermentation method is capable of producing 60 g/L to 140 g/L of malonate thereof over a time in a range of 20 hours to 100 hours.

Aspect 28 provides the fermentation method of any one of Aspects 1-27, wherein the malonate comprises:
the compound of formula I:

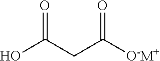

wherein M is a Group I alkali metal or ammonium,
optionally comprises the compound of formula II:

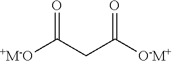

wherein M is a Group I alkali metal or ammonium, and
optionally comprises malonic acid.

Aspect 29 provides the fermentation method of Aspect 28, wherein the malonate comprises at least 50 percent (for example at least 55 percent, at least 60 percent) by weight of the compound of formula I; no greater than 25 percent (for example no greater than 20 percent, no greater than 15 percent, no greater 10 percent, or not greater 5 percent) by weight of the compound of formula II; and less than 50 percent (less than 45 percent, less than 40 percent, less than 35 percent, less than 30 percent) by weight malonic acid.

Aspect 30 provides the fermentation method of Aspect 28, wherein the malonate comprising at least 40 wt % (for example, at least 45 wt %, at least 60 wt % (for example from 40 wt % to 90 wt %, from 60 wt % to 90 wt %) of the compound of formula 1; less than 55 wt % (for example, less than 50 wt %, less than 15 wt % for example, from 1 wt % to 55 wt %, from 1 wt % to 15 wt %) of malonic acid; and less than 25 wt % (for example, less than 20 wt %, less than 17 wt % (for example from 0.05 wt % to 20 wt %, from 1 wt % to 17 wt %)) of the compound of formula II.

Aspect 31 provides the fermentation method of Aspects 1-30, wherein the fermentation method further comprises recovering malonate and removing biomass from the fermentation broth before recovering the malonate.

Aspect 32 provides the fermentation method of any of Aspects 1-31, wherein the base other than a calcium containing base comprises a base containing a potassium.

Aspect 33 provides the fermentation method of any one of Aspects 1-32, wherein the fermentation method comprises simultaneous saccharification and fermentation.

Aspect 34 provides the fermentation method of any one of Aspects 1-33, wherein the method is carried out in aerobic, microaerobic or anaerobic conditions.

Aspect 35 provides the fermentation method of any one of Aspects 1-34, wherein malonate is produced at a rate of at least 0.8 g L⁻¹ h⁻¹ to 3 g L⁻¹ h⁻¹.

Aspect 36 provides the fermentation method of any one of Aspects 1-35, wherein malonate is produced at a rate of at least 1 g L⁻¹ h⁻¹ to 3 g L⁻¹ h⁻¹.

Aspect 37 provides the fermentation method of any one of Aspects 1-36, wherein less than 5 g/L of ethanol is produced after 36 hours.

Aspect 38 provides an engineered microorganism capable of producing malonate, the engineered microorganism comprising:
a heterologous malonate-semialdehyde dehydrogenase that comprises at least 90% sequence identity to any one of SEQ ID No: 27, wherein the engineered microorganism is capable of producing about at least 60 g/L at a pH of xxx.

Aspect 39 provides the engineered microorganism of Aspect 38 wherein heterologous malonate-semialdehyde dehydrogenase that comprises at least 95% sequence identity to any one of SEQ ID No: 27.

Aspect 40 provides the engineered microorganism of any one of Aspects 38 or 39, wherein the engineered microorganism is capable of producing about at least 60 g/L at a pH in a range of 2.5 to 6.2, for example from 2.5 to 5.0, from 2.5 to 4.5, from 3.0 to 4.3, or from 3.0 to 4.0.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

1. A fermentation method for producing malonate, the method comprising:
a) culturing a microorganism in the presence of a fermentation medium, the fermentation medium comprising:
at least one carbon source;
calcium ions ranging from 0.05 g/L to 1.6 g/L; and
a base other than a calcium containing base; and
b) producing at least 40 grams/liter of malonate (for example, in a range of from 40 grams/liter to 200 grams/liter, from 60 grams/liter to 150 grams/liter, from 80 grams/liter to 150 grams/liter, from 90 grams/liter to 150 grams/liter, or from 100 grams/liter to 150 grams/liter malonate), wherein optionally, the malonate is produced at a rate of at least 0.3 g L⁻¹ h⁻¹ to 5 g L⁻¹ h⁻¹.

2. (canceled)

3. (canceled)

4. The fermentation method of claim 3, wherein the calcium ions are provided from a calcium ion containing compound that is added to the fermentation medium.

5. The fermentation method of claim 4, wherein the calcium ion containing compound comprises CaCO₃, CaCl₂, Ca(OH)₂, or a mixture thereof.

6. The fermentation method of claim 5, wherein the calcium ion containing compound comprises Ca(OH)₂.

7. The fermentation method of claim 5, wherein the calcium ion is provided to the fermentation medium at a concentration in a range of 0.1 g/L to 1.2 g/L.

8. The fermentation method of claim 6, wherein the calcium ion is provided to the fermentation medium at a concentration in a range of 0.1 g/L to 0.5 g/L.

9. The fermentation method of claim 1, wherein the calcium ions are present at a start of the fermentation method.

10. The fermentation method of claim 1, wherein the calcium ions are added during the fermentation method.

11. The fermentation method of claim 1, wherein an oxygen uptake rate of the microorganism during the fermentation method is in a range of from 25 mmol L⁻¹ h⁻¹ to 60 mmol L⁻¹ h⁻¹.

12. (canceled)

13. The fermentation method of claim 1, wherein the base comprises KOH, NaOH, LiOH, and ammonia, or a mixture thereof.

14. The fermentation method of claim 1, wherein the base comprises KOH.

15. The fermentation method of claim 13, wherein the base is in a range of from 0.8 wt % to 15 wt % of the fermentation medium.

16. The fermentation method of claim 14, wherein the base is in a range of from 1.6 wt % to 8 wt % of the fermentation medium.

17. The fermentation method of claim 1, wherein a pH during fermentation is in a range of 2.5 to 7.

18. The fermentation method of claim 1, wherein a pH during fermentation is in a range of 2.5 to 6.2, example from 2.5 to 5.0, from 2.5 to 4.5, from 3.0 to 4.0, from 3.0 to 3.5, and in some instances where a higher pH is desirable from 3.5 to 6.0, from 3.7 to 5.3.

19. The fermentation method of claim 18, wherein the base functions to control a pH of the fermentation medium.

20. (canceled)

21. The fermentation method of claim 1, wherein the microorganism comprises an engineered microorganism.

22. The fermentation method of claim 21, wherein the microorganism comprises yeast, for example, a genetically modified Crabtree negative yeast.

23. The fermentation method of claim 22, wherein the yeast comprises at least one of the Issatchenkia orientalis or Pichia fermentans clades, preferably Issatchenkia orientalis.

24. (canceled)

25. (canceled)

26. The fermentation method of claim 1, wherein the fermentation method is capable of producing 40 g/L to 200 g/L of malonate over a time in a range of 15 hours to 200 hours.

27. (canceled)

28. The fermentation method of claim 1, wherein the malonate comprises:
the compound of formula I: 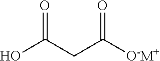
wherein M is a Group I alkali metal or ammonium,
optionally comprises the compound of formula II: 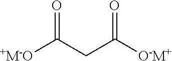
wherein M is a Group I alkali metal or ammonium, and
optionally comprises malonic acid.

29-40. (canceled)
